(19) United States
(12) Patent Application Publication
(10) Pub. No.: US 2022/0285017 A1
(43) Pub. Date: Sep. 8, 2022

(54) OPTIMIZED OPERATING ROOM BLOCK MANAGEMENT AND RELATED SYSTEMS AND METHODS

(71) Applicant: QuiviQ, Inc., Bellevue, WA (US)

(72) Inventors: Michael Larson, Mercer Island, WA (US); Charles Bird, Sammamish, WA (US); Katrina Mikofalvy, Seattle, WA (US); Marc Diamond, Renton, WA (US); Nathan Fong, Seattle, WA (US); Glenn Doherty, Port Ludlow, WA (US)

(21) Appl. No.: 17/689,835

(22) Filed: Mar. 8, 2022

Related U.S. Application Data
(60) Provisional application No. 63/157,946, filed on Mar. 8, 2021.

(51) Int. Cl. G16H 40/20; G16H 50/20

(52) U.S. Cl. CPC G16H 40/20; G16H 50/20

(57) ABSTRACT

Systems and methods for improving operating room utilization are disclosed herein. Ins some implementations, the method includes retrieving historical statistics of usage rates for an operating room, predicting an estimate of how much time will be unused during a future time block based on the historical information and a planned schedule, and generating a recommendation for the unused time in the estimate. In some implementations, predicting the estimate includes constructing one or more regression models based on the historical statistics. In such implementations, each of the one or more regression models can identify a relationship between the planned usage rate and the actual usage rate based on a different modeling technique. Once the estimate is obtained, the recommendation for the unused time can be based at least partially on an identification of one or more intervals in a future time block that are associated with an open operating room.

### CROSS-REFERENCE TO RELATED APPLICATION(S)

The present application claims the benefit of U.S. Provisional Patent Application No. 63/157,946, filed Mar. 8, 2021, the entirety of which is incorporated herein by reference.

### TECHNICAL FIELD

The present technology is generally directed to systems and methods for improving the utilization rate of operating rooms. More specifically, aspects of the present technology relate to controlling operating room schedules to maximize the efficiency of operating room usage as well as the usage of related operating room personnel.

### BACKGROUND

Hospitals and other health care systems typically rely on block schedules for managing complex operating room utilization. Block time is when a specific surgeon (or surgical group) is assigned an operating room for a partial day or full day in advance. That surgeon owns that particular operating room for that block of time and then schedules surgical procedures into that block of time.

### BRIEF DESCRIPTION OF THE DRAWINGS

FIG. 1 is an architecture diagram in which aspects of the disclosed technology are incorporated.

FIG. 2 is a high level flow diagram depicting how an optimized operating room block management system identifies the status of block recommendations to proactively release free blocks that are unlikely to be used.

FIGS. 3A-3E are flow diagrams illustrating an overview of an environment in which some implementations of the disclosed technology can operate and how users are notified of workflow changes.

FIG. 4 is a flow diagram illustrating a process performed by some implementations of an optimized operating room block management system.

FIGS. 5A-5K are example user interfaces utilized by some implementations of an optimized operating room block management system for operating room schedule management.

FIGS. 6A-6B are example user interfaces utilized by some implementations of an optimized operating room block management system for operating room request management.

FIGS. 7A-7D are example user interfaces utilized by some implementations of an optimized operating room block management system for performing analytics on the operating room data.

FIG. 8 is a block diagram that illustrates an example of a computer system 800 in which at least some operations described herein can be implemented.

FIG. 9 is a system diagram illustrating an example of a computing environment in which the disclosed system operates in some implementations of the present technology.

FIG. 10 is a flow diagram of a process for improving operating room utilization in accordance with some implementations of the present technology.

The technologies described herein will become more apparent to those skilled in the art from a study of the Detailed Description in conjunction with the drawings. Implementations are illustrated by way of example and not limitation in the drawings, in which like references can indicate similar elements. While the drawings depict various implementations for the purpose of illustration, those skilled in the art will recognize that alternative implementations can be employed without departing from the principles of the technologies. Accordingly, while specific implementations are shown in the drawings, the technology is amenable to various modifications.

The drawings have not necessarily been drawn to scale. Similarly, some components and/or operations can be separated into different blocks or combined into a single block for the purpose of discussion of some of the implementations of the present technology. Moreover, while the technology is amenable to various modifications and alternative forms, specific implementations have been shown by way of example in the drawings and are described in detail below. The intention, however, is not to limit the technology to the particular implementations described.

### DETAILED DESCRIPTION

### Overview

Existing systems for booking operating room resources in block times suffer from several deficiencies. When a surgeon does not fill their block with cases, then the operating room loses money—some estimates put the opportunity cost of an idle operating room at $15-20 per minute after factoring in staff time, equipment, administrative costs, etc. In addition, patients in need of care may find themselves waiting for treatment while critical resources go unused. Conversely, if blocks are booked solid, then there may be insufficient capacity to handle emergency cases. For example, a trauma patient who shows up in the emergency department or a patient in the ICU who needs an exploratory laparotomy may require urgent access to operating room resources. In order to accommodate these surgical cases that cannot be scheduled weeks or months in advance, there has to be sufficient “open time” when surgeons can add on cases on relatively short notice.

Further, block times are not all the same duration. For example, an otolaryngologist who does a lot of tonsillectomies and septoplasties may be able to do a surgery every 30 minutes, and so a 4-hour block may be appropriate. In another example, an otolaryngologist who does complex neck cancer surgeries with flap creation may be doing procedures that can take all day, and so an 8-hour block may be appropriate. In addition, some surgeons can warrant having 2 operating rooms simultaneously blocked, such as the otolaryngologist doing tonsillectomies and septoplasties, who can be doing a surgery in one room in about the time it takes to clean another room, allowing him/her to efficiently go back and forth between the two rooms. The otolaryngologist doing the major neck cancer surgery for 8 hours only needs one operating room per block time.

Open time can be created by either purposefully keeping some operating rooms out of the block time schedule so that they are never blocked out or open time can be created by “releasing” block time when a surgeon does not have a full calendar of cases scheduled for that particular block. For some specialties that normally schedule their cases electively weeks or months in the future (e.g., for joint replacement surgery, it may be appropriate to release a surgeon's block weeks in advance if he/she has no cases on the schedule by that time). However, for other specialties, it may not be possible to release a block more than a day or two in the future (e.g., for neurosurgery, once a brain tumor or aneurysm is diagnosed, it has to be operated on within a few days). Therefore, the hospital has to have the right balance between block time and open time.

When a surgeon does not perform any surgeries during a given block, it is generally straightforward to define that block as unutilized. However, conventional systems can struggle to evaluate the utilization of more complicated schedules, such as for a surgeon who schedules two 1-hour surgeries in a 6-hour block but generally completes slower than expected (e.g., typically takes an hour and twenty minutes per medical procedure (e.g., surgical operation, diagnostic operations, and the like)), or a surgeon who schedules six 1-hour surgeries in the 6-hour block but generally completes faster than expected (e.g., completes each procedure in about 30 minutes). Accurately distinguishing between unutilized versus underutilized blocks in this setting can be extremely difficult based on the variance in actual operating time from a planned schedule, let alone accounting for desired open time in each of the blocks for emergency procedures. Currently, the process by which unused blocks (and/or any blocks can be identified as under-utilized) are released to other potential users is manual, complicated and inefficient, resulting in many blocks going unused. As a result, operating room resources are wasted, and the increased cost of operation are generally passed on to patients.

The implementations disclosed herein describe systems and methods for improving and/or optimizing operating room utilization, as well as improving and/or optimizing the utilization of related resources (e.g., personnel such as surgeons, surgical support staff, cleaning staff, and the like; operating room equipment, power, and the like; post-surgery resources such as recovery spaces, post-surgery staff, and the like; and/or various other related resources). As one example, the methods and systems are described herein for.

To overcome these technical deficiencies in conventional systems, systems and methods are disclosed herein can generate one or more models based on historical planned usage rates for one or more operating rooms (e.g., the time the operating room(s) were scheduled to be occupied for various procedures, pre-procedure preparation, post-procedure cleaning, and the like) as well as historical actual usage rates (e.g., the time the operating room(s) were actually occupied after canceled procedures, variances in operating times for procedures, emergency procedures, and the like); uses the models to predict estimates for future usage rates for the operating room(s); identifies open intervals during which the operating room will not be utilized (e.g., not being used for a procedure and/or not transitioning between procedures); then generates one or more recommendations for what to do with the open intervals. Because the models are constructed based on historical data for the operating room, they are able to accurately predict actual future usage rates for the operating room to more accurately plan future procedures to avoid both over-scheduling (which can lead to no operating rooms being available for emergency procedures) and under-scheduling (leading to inefficient usages of the operating rooms, increased medical costs passed to patients, and the like). For example, in some implementations, the systems and methods disclosed herein aim to eliminate inefficiency by harvesting unused blocks of time in operating rooms. The optimized operating room block management system predicts, far enough in advance, which blocks assigned to a particular surgeon/clinic/specialty are unlikely to be used and aims to either redirect them to individuals/groups that may be in need of surgical blocks or close the operating rooms for short periods of time to reduce operational costs. While doing so, the systems and methods disclosed herein account for unplanned occupancy and/or unplanned open operating rooms while before generating recommendations for changes. In addition, the recommendations can identify changes that improve operating room utilization in real-time, then facilitate changes to an operating room schedule based on the recommendations. As a result, the systems and methods disclosed herein can accomplish operating room management in real-time and based on objective determinations.

In a specific, non-limiting example, a method according to the present technology can include retrieving, receiving, and/or recording (sometimes referred to collectively herein as “retrieving”) statistics indicative of usage rates for an operating room, where the statistics include historical information related to one or more individual time blocks (e.g., blocks in a calendar for the operating room); predicting an estimate of how much time will be unused during a future time block based on the historical information and a planned schedule; and generating a recommendation for the unused time in the estimate. In some implementations, the historical information can include, for one or more operating rooms, planned usage rates for individual previous time blocks; actual usage rates for the previous time blocks; metadata related to the planned and/or actual usage rates (e.g., an indication of which medical practitioners were scheduled, which practitioners actually used the operating room, an accounting for a difference between the planned and actual time (e.g., emergency procedures scheduled, procedures taking longer or shorter than planned, and the like); requested changes from patients; and/or any other suitable metadata). In some implementations, predicting the estimate includes constructing one or more regression models based on the historical statistics of usage rates. In such implementations, each of the one or more regression models can identify a relationship between the planned usage rate and the actual usage rate based on a different modeling technique. Predicting the estimate can also include identifying, from the one or more regression models, a best fit model for the historical statistics, and applying the best fit model (or any subset of the one or more regression models) to the planned schedule (e.g., planned usage rates, such as planned procedure schedules). Once the estimate is obtained, the recommendation for the unused time can be based at least partially on an identification of one or more intervals in a future time block that are associated with an open operating room (e.g., predicted dead time, windows without a scheduled procedure and/or predicted to open up, time predicted to be open based on variances in procedure time from planned timed, and the like).

In various implementations, the recommendation can include: releasing one or more of the identified intervals for additional procedures in the operation room; rescheduling one or more procedures during the future block into one or more of the identified intervals to increase an amount of continuously available time during the future block; rescheduling one or more procedures during the future block into one or more of the identified intervals to reduce down time for operating room personnel (e.g., surgeons, nurses, anesthesiologists, radiologists, surgical assistants, cleaning personnel, and the like) the future block; closing the operating room during one or more of the identified intervals; and/or various other suitable changes to the operating room schedule.

In some implementations, generating the recommendation includes determining an amount of available time in each of the identified intervals (e.g., thirty minutes, 50 minutes, 1-hour, 1-hour and twenty-five minutes, 2-hours, and/or any other amount of time). When the amount of available time is above a predetermined minimum threshold (e.g., thirty minutes, 1-hour, two-hours, etc.), the recommendation can include releasing the identified interval for booking for another procedure. Conversely, when the amount of available time is below the predetermined minimum threshold, the recommendation can include closing the operating room during the identified interval and/or releasing personnel assigned to the operating room during the interval (e.g., allowing nurses to be scheduled in another operating room and/or take a scheduled break). In some implementations, generating the recommendation includes identifying adjacent intervals in the identified intervals and combining the time in adjacent intervals. Accordingly, the amount of available time in combined intervals can be increased, which can allow the method to identify additional intervals that can be released for another procedure. In some implementations, determining the amount of available time includes determining the smaller of an amount of available time identified by the estimate and a maximum time for the interval (e.g., thereby avoiding over-estimation errors).

In some implementations, generating the recommendation includes determining whether any of the identified intervals is the first interval of the future block (e.g., the beginning of the future time block). When one of the identified intervals is the first interval in the block, generating the recommendation can include determining an amount of time available during the first interval. When the amount of available time is above a predetermined threshold, the recommendation can include releasing the first interval to allow another procedure to be booked. Conversely, when the amount of available time is below the predetermined threshold, the recommendation includes closing the operating room during the first interval (e.g., thereby allowing operating room personnel to be reassigned and/or scheduled to arrive later). In some implementations, recommendations are interdependent recommendations generated for a plurality of operating rooms. For example, when an interval is identified as available for a procedure in a first operating room, a procedure from a second operating room can be recommended to be moved to the first operating room to reduce operating costs of the second operating room, free additional time in the second operating room for more substantial (e.g., longer) procedures, and/or to capitalize on efficient resource deployment between the first and second operating rooms.

In some implementations, generating the recommendation includes determining whether any of the identified intervals is a last interval of the future time block (e.g., the end of the future time block). When one of the identified intervals is the last interval, generating the recommendation can include determining an amount of time available during the last interval. In some implementations, determining the amount of time available during the last interval includes: (1) identifying the predicted ending time for the last planned procedure in the future block, (2) identifying the available time between the last planned procedure and the last interval (e.g., the unused time at the end of the interval for the planned procedure), and (3) adding at least a portion of the time difference to the last interval to increase the amount of available time. That is, determining the amount of time available during the last interval can include expanding the last interval into unused time in a previous interval to increase the amount of available time. When the amount of available time is above a predetermined threshold, the recommendation can include releasing the last interval to allow another procedure to be booked. When the amount of available time is below the predetermined threshold, the recommendation can include closing the operating room during the last interval (e.g., thereby allowing operating room personnel to be reassigned and/or scheduled to depart sooner).

In some implementations, the method includes sending the recommendation to at least of a surgeon, a scheduling manager, an administrator associated with the operating room, a block owner, and a patient.

In some implementations, the method includes displaying the recommendation to a user (e.g., through a user interface, displaying the recommendation to a schedule manager, a surgeon, a patient, an operating room manager, and the like). The method can also include receiving an indication from the user of an approval or a denial of the recommendation. When the recommendation is approved, the method can include updating the planned usage rates during the future block based on the recommendation. When the recommendation is denied, the method can include generating a new recommendation for the unused time in the estimate.

In some implementations, the estimate of unused time includes a plurality of sub-estimates each associated with a different level of abstraction for usage of the operating room (e.g., usage rates by medical personnel such as surgeons, doctors, anesthesiologists, radiologists, and the like; usage rates by all operating room staff, such as nurses, pre-operation personnel, post-operation personnel, and the like; and/or usage rates across all facility personnel). Additionally, or alternatively, the recommendation based on the unused time can include recommendations at a variety of levels of abstraction. In some implementations, the recommendation is further based at least partially on a predetermined required amount of unused time for the operating room.

The following description and drawings are illustrative and are not to be construed as limiting. Numerous specific details are described to provide a thorough understanding of the disclosure. Further, the following description provides certain specific details for a thorough understanding and enabling description of these examples. One skilled in the relevant technology will understand, however, that the invention may be practiced without many of these details. Likewise, one skilled in the relevant technology will also understand that the invention may include many other obvious features not described in detail herein. Additionally, some well-known structures or functions may not be shown or described in detail below, to avoid unnecessarily obscuring the relevant descriptions of the various examples.

However, in certain instances, well-known or conventional details are not described in order to avoid obscuring the description. Reference in this specification to “one implementation” or “an implementation” means that a particular feature, structure, or characteristic described in connection with the implementation is included in at least one implementation of the disclosure. The appearances of the phrase “in one implementation” in various places in the specification are not necessarily all referring to the same implementation, nor are separate or alternative implementations mutually exclusive of other implementations. Moreover, various features are described which may be exhibited by some implementations and not by others. Similarly, various requirements are described which may be requirements for some implementations but no other implementations.

### Description of the Figures

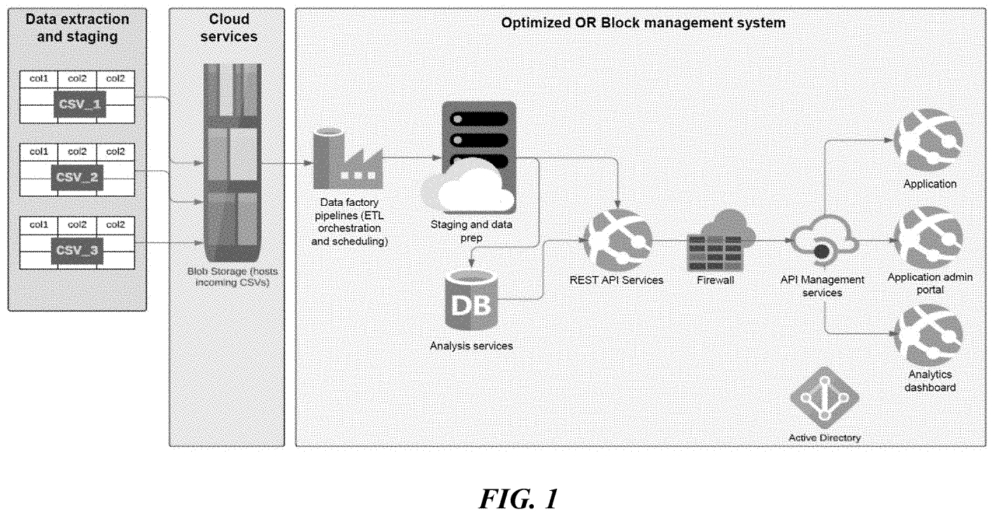

FIG. 1 is an architecture diagram in which aspects of the disclosed technology are incorporated. One of skill in the art will understand that the specific technologies depicted in FIG. 1 are examples and that other equivalent technologies can be used instead. For example, while FIG. 1 shows extraction and staging using csv formatted data, other equivalent data storage formats (e.g., markup language files, database tables, etc.) can be used instead or in addition to csv formatted data. Similarly, while the staging and data preparation component(s) are depicted as SQL databases, other database types (e.g., dB2, etc.) can also be used.

The disclosed technology can be implemented in a batch and/or event driven manner. For example, in some implementations, a periodic job (e.g., nightly job) is executed to process one or more bookings and/or block state changes. In some implementations, one or more events can trigger the execution of processes (e.g., analytics computations) related to the optimized operating room block management system. Examples of events include, but are not limited to, new bookings, cancelled bookings, edited booking, other edits to schedule/calendar, and so on. In some examples of event driven execution, the disclosed technology does not perform the extraction of information based on files; instead, it can process the individual pieces of information that have changed.

### I. Block Release Recommendation Process

At a high level, the systems and methods disclosed herein require two steps to generate recommendations for adjustments to one or more intervals in a block of time in order to improve (and/or optimize) the usage rate for an operating room: (1) the generation of an estimate of the unused time in that block that will be available during the given block, broken into one or more intervals of time, and (2) the use of the estimate, in conjunction with the currently booked time intervals, to generate a final recommendation that accounts for open and/or incomplete intervals. These two steps, and the method therein, are sometimes referred to herein as the block release recommendation process.

A. Generation of the Estimate of Unused Time in a Future Block of Time

An example implementation for generating unused time estimates is described below. The estimate of unused time on day of procedure can be provided at multiple levels of abstraction and/or for a specific number of days before procedure. For example, the following three levels of abstraction can be used: (1) for the specific surgeon or surgical group that owns the block, (2) for the surgical service line of the block (e.g., Orthopedics), and (3) for the entire population.

At each level of abstraction, statistics are collected from historical block usage to construct frequency records in one or more data formats (e.g., table(s), comma separated file(s), markup language formatted file(s), etc.) of booked time for a specific number of days before procedure. An example table can comprise rows and columns, where each row and each column corresponds to a specific time range. For example, procedures and/or the time block for the operating room can each be broken into time ranges (e.g., intervals). In a specific, non-limiting example, columns can correspond to intervals for procedures booked on the day of a procedure (e.g., procedures schedule in an emergency and/or instant treat basis), while each row can correspond to intervals for procedures booked in advance of the procedure date (e.g., planned procedures). In this example, a row can represent the time range of 150 to 300 minutes and a column can represent the time range from 300 to 450 minutes. The cell at the intersection of this row and each column contains the count of cases in the historical data, where the booked time at the specific number of days before procedure is in the range of 150 to 300 minutes and the number of actual time booked at day of procedure is in the range of 300 to 450 minutes. The frequency records (e.g., tables) can be specific to each level of abstraction. In implementations where procedure cancellations are not used, the table will have zeros below the diagonal.

Given the frequency records thus constructed for a specific number of days before procedure and level of abstraction, one or more models are constructed to explain the observed frequencies. The basis of the model can be one or more regressions. For example, in some implementations, the basis of the model is two types of regressions: one is based on a Poisson model and the other is based on a Negative Binomial model. In various other implementations, the regression models can include a Bayesian model, a Lasso model, a Polynomial model, and/or various other suitable regression models. Regressions for both models are executed and the model type that results in the best fit is selected. The result of the modeling process is an estimate of the probability for the day of procedure time for each column and the corresponding time range. This probability is used to compute an estimate of the day of procedure booked time as a function of the booked time range for the number of days before procedure. In some implementations, only frequency tables with a threshold number of samples (e.g., 50 or more samples) are used to generate statistics.

The resulting functions can be of the form:

fₛ(r,d,s)=Eₛ[t_{b}], (1)

fₛ(r,d,S)=Eₛ[t_{b}], and (2)

fₚ(r,d)=Eₚ[t_{b}] (3)

where, fₛ(r, d, s) is the estimate at the surgeon or surgical group level, r corresponds to the number time range for the amount of time booked at d days before procedure, and s is the specific surgeon or surgical group; fₛ(r, d, S) is the estimate at the surgical service level, r corresponds to the number time range for the amount of time booked at d days before procedure, and S is the specific surgical level; and fₚ(r, d) is the estimate over the entire population, and r corresponds to the number time range for the amount of time booked at d days before procedure.

As an example, the date ranges for both rows and columns of this table can be: [0, 0], (0, 150], (150, 300], (300, 450], . . . , (1200, 1350], (1350, and up]. In this example, times are in minutes.

B. Generation of the Recommendation Based on the Estimate of Unused Time

An example implementation for generating unused time estimates is described below. All blocks within the specified number of days before are examined individually, and a recommendation is potentially generated. Let B be a specific block, s be the surgeon or surgical group associated with B, and S be the surgical service line associated with B. The system can generate an estimate of day of procedure booked time as follows:

(1) If fₛ(r, d, s) exists for the given s, then use fₛ(r, d, s)=Eₛ[t_{b}] as the estimate of day 0 booked time, otherwise; (2) if fₛ(r, d, S) exists for the given S, then use fₛ (r, d, S)=Eₛ[t_{b}] as the estimate of day 0 booked time, otherwise; (3) use fₚ(r, d)=Eₚ[t_{b}] as the estimate of day 0 booked time. It will be understood that that fₛ(r, d, s) and/or fₛ(r, d, S) will exist only if there are sufficient samples to generate the function. The resulting estimate, based on the historical statistics and the model generations above, can be called Eₛ.

Associated with block B at d days before procedure, will be a block start date, bₛ and time and a block end date and time, bₑ and a set of time intervals I_{U}={i₁^{u}, i₂^{u} . . . , i_{M}^{u}} which are booked for procedure. From this a set of open time intervals I_{U}={i₁^{o}, i₂^{o}, . . . , i_{N}^{o}} is generated. If I_{U} is empty, then I_{O} contains a single interval [bₛ, bₑ]. Let t_{[max]} be the length in time of the largest open interval. Note that multiple open time intervals may often be combined into larger time intervals by adjusting the scheduled procedure times and sequencing. The amount of time recommended for release is:

t_{R}=min{E_{S},t_{[max]}} (4)

That is, the lesser of the amount of time implied by the estimate E_{S} and t_{[max]}. The process of constructing a specific interval with t_{R} time can then be compared against a threshold value relevant to help generate the any recommendations. Purely by way of example, when t_{R}<threshold value (e.g., 120 minutes) then no change is recommended for the interval in the block (e.g., the recommendation is to maintain the schedule as-is).

As another example, when there is open space at the beginning of the block that is unused and the amount of time open at the beginning of the day is at least t_{R}, then the system provides a recommended release interval value computed as [bₛ, bₛ+tᵣ]. The system can find the first interval, iⱼ^{o} from the set I_{O} which is at least t_{R} long, then provide a recommended release interval. For example, the recommended release interval can be computed as [i_{[j,e]}^{o}, i_{[j,e]}^{o}+tᵣ], where is the endpoint of the interval iⱼ^{o}.

As yet another example, when there is open space at the end of the block that is unused and the amount of time open at the end of the day is at least t_{R}, then the system provides a recommended release interval value computed as [i_{[M,e]}^{o}, i_{[M,e]}^{o}+tᵣ], where i_{[M,e]}^{o} is the ending time of the last used interval associated with B.

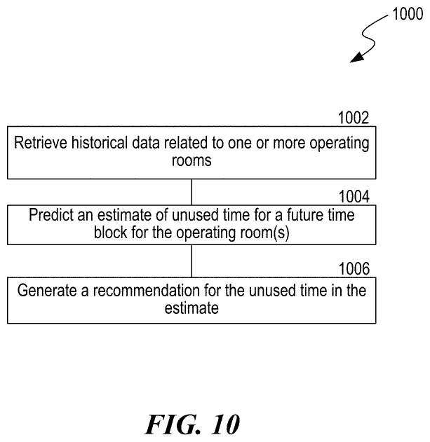

FIG. 10 is a flow diagram of a process 1000 for improving operating room utilization based on the block release recommendation process described above in accordance with some implementations of the present technology. The process 1000 begins at block 1002 with retrieving historical data related to one or more operating rooms. The historical statistics can include planned usage rates for the operating room, actual usage rates, and/or any related data and/or metadata (e.g., metadata explaining the difference between the planned and actual usage rates). In some implementations, the historical statistics can include statistics for a variety of levels of abstraction (e.g., surgeon statistics, staff statistics, hospital statistics, and the like).

At block 1004, the process 1000 includes predicting an estimate of unused time for a future time block. To make the prediction, the process 1000 can construct one or more regression models based on the historical statistics for the operating room. As discussed above, each of the one or more regression models can identify a relationship between the planned usage rate and the actual usage rate and be based on an alternative modeling technique. The process 1000 can then identify a model with the best fit for the historical statistics from the one or more models. The process 1000 can then apply the best fit model to planned future usage rates for the operating room, thereby generating an estimate of the actual future usage rates. In some implementations, the process 1000 applies each of the one or more regression models to the future usage rates for the operating room, thereby generating a variety of estimates. In some implementations, the process 1000 can predict a plurality of estimates, each associated with a different level of abstraction for usage rates (e.g., surgeon usage rates, staff usage rates, hospital usage rates, and the like).

At block 1006, the process includes generating a recommendation for the unused time in the estimate. The recommendation can be based at least partially on an identification of one or more intervals that are associated with an open operating room; one or more reasons past recommendations were denied; one or more pending requests for procedures and/or changes to the operating room schedule; one or more stated goals for the operating room (e.g., a predetermined required amount of unused time for the operating room to ensure adequate room for emergency procedures); and the like.

### II. Example Systems, Methods, and User Interfaces Related to Generated Recommendations

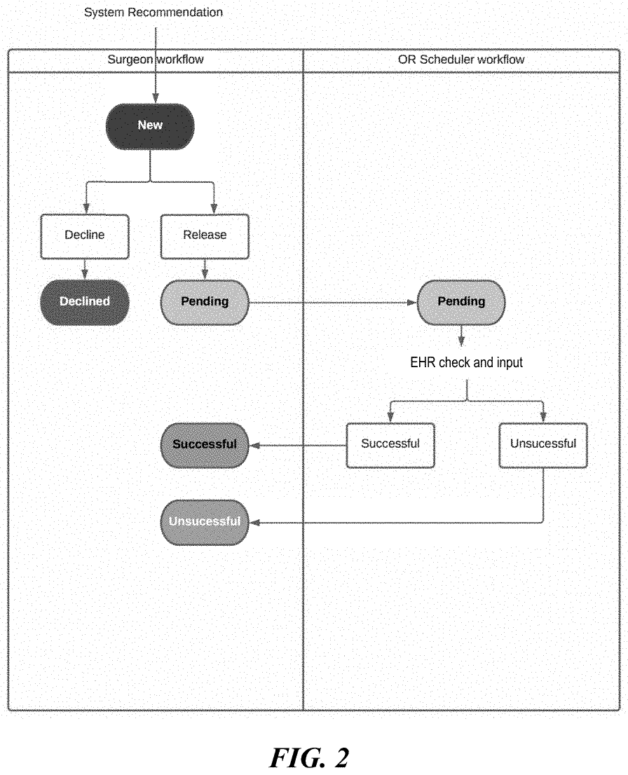

FIG. 2 is a high level flow diagram depicting how the optimized operating room management system identifies the status of recommendations to proactively release free intervals within blocks that are unlikely to be used, make changes to planned procedures (sometimes also referred to herein as “operations”) within blocks to free additional time within a block and/or optimize resources, and/or add operations into an existing schedule. As illustrated the recommendation release process pushes “new” recommendations to the clinic-side users who can then decline the recommendation (moving to a “declined” status) or release (e.g., approve and forward) the recommendation (moving to a “pending” status). A “pending” recommendation is sent to the hospital-side users who log the release in a schedule management system, marking the status as “successful” to the clinic-side users if the recommendation was able to be released (e.g., if the recommendation is fully accepted) or “unsuccessful” to the clinic-side users if the recommendation was not able to be released. In some implementations, the successful and/or unsuccessful release of a recommendation is dependent on approval from administrators, such as a hospital manager. In some implementations, the successful and/or unsuccessful release of a recommendation is dependent on acceptance (or acknowledgements) to a schedule change from all staff involved.

The output of the block release recommendation process is a set of blocks and for each element of this set, a specific interval within that block to recommend for release. The process for generating block release interval recommendations can be run periodically (e.g., nightly or triggered by events) and the specific recommendations can be stored in one or more databases. The information can then be used to provide recommendations (for example, to the block owners, administrators, etc.) for release of the specific intervals.

In some implementations, a subset of the blocks are processed at periodic intervals (e.g., daily, nightly, weekly, bi-weekly, monthly, quarterly, etc.) and/or when particular events occur. For example, not all blocks in the system are processed during a nightly run. Only those that satisfy one or more criteria are considered (e.g., blocks with a procedure date that is within a specific range of number of days of the current date are considered). Typically, the system will consider only those blocks that have procedure dates that are within a configurable date range (e.g., 14 to 21 days from the current date (date of the run)), although blocks from additional date ranges can be considered as well. For each block considered, the process described below is applied.

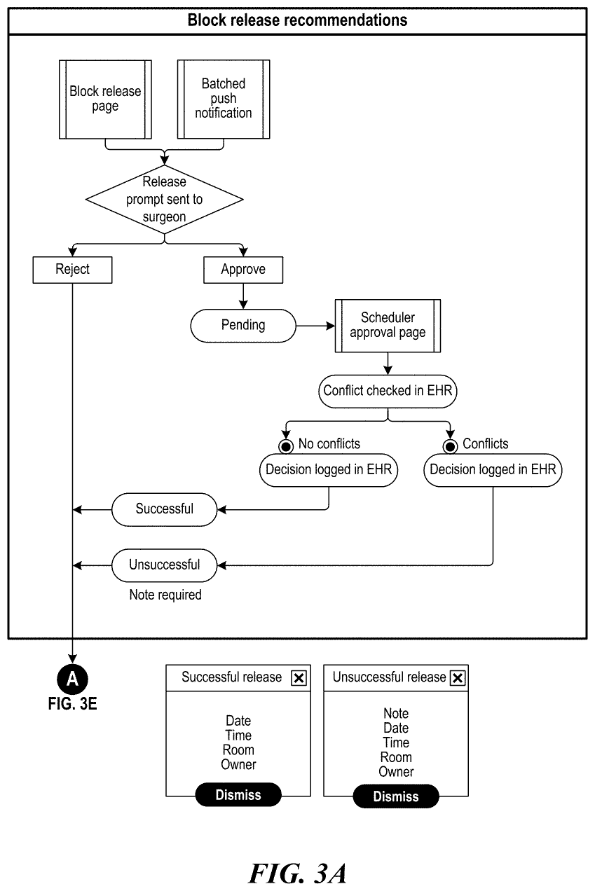

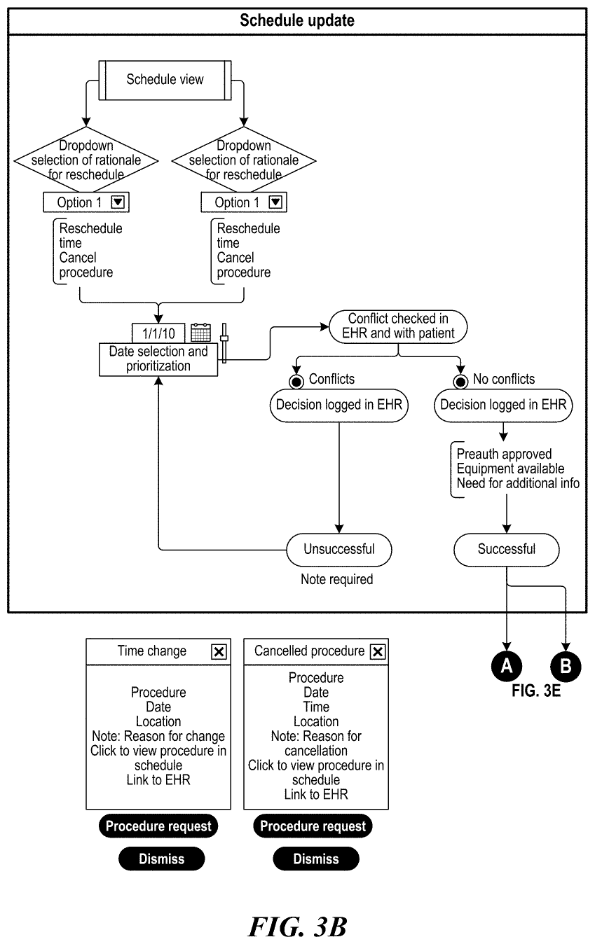

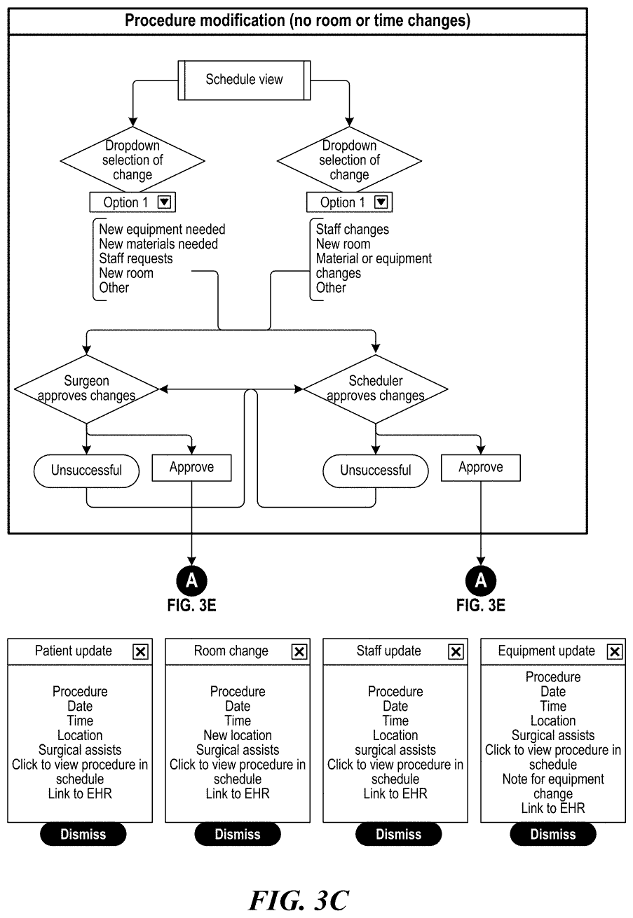

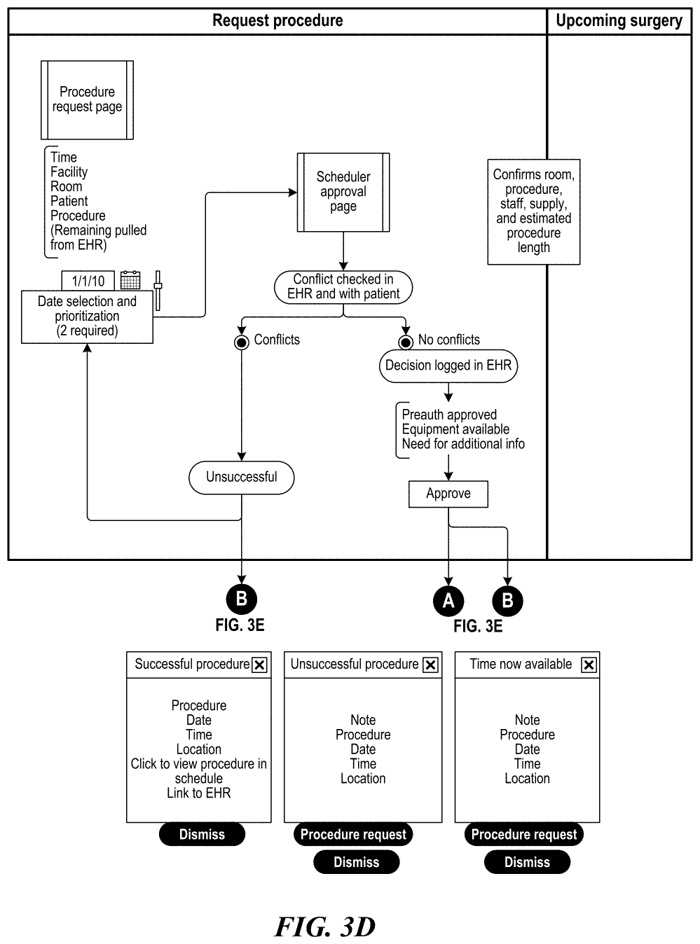

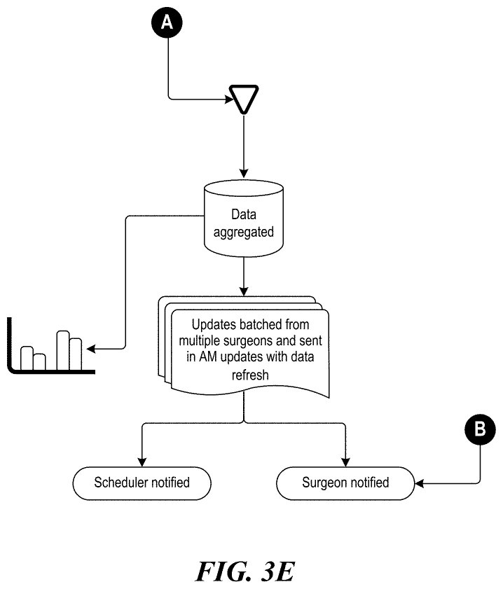

FIGS. 3A-3E are block diagrams illustrating an overview of an environment in which some implementations of the disclosed technology can operate and how users are notified of workflow changes. The illustrated diagrams incorporate both release functionality as well the ability to schedule procedures within and outside of blocks (e.g., depending on available intervals).

For example, in FIG. 3A, a recommendation is sent to a surgeon (e.g., after the process in FIG. 10 completes for one or more recommended changes), allowing a surgeon to reject or approve a recommendation. If rejected, the process moves on to consider alternative changes and/or changes for future blocks. If accepted, the process updates the status of the recommendation to pending and forwards the recommendation to a scheduler approval page (e.g., a user interface provided to a schedule manager) for review. The process then includes checking for conflicts with the recommendation (e.g., conflicts with the surgeon's schedule unrelated to the operating room, such as meetings, other scheduled procedures in another operating room or on another campus, and the like). When conflicts are detected, the conflict is recorded for later review and the recommendation status is updated to unsuccessful. As illustrated in FIG. 3A, an update to unsuccessful for the recommendation can require a note that indicates a reason for the failure (e.g., an indication of the identified conflict). Conversely, if no conflicts are detected, the conflict is recorded for later review and the recommendation status is updated to successful.

FIG. 3B illustrates a process for reviewing and/or accepting a recommendation to reschedule and/or cancel a procedure to improve an operating room's efficiency (e.g., moving an operation forward to provide additional time after the operation for a new operation; canceling an operation that is determined to be an inefficient use of time; and the like). Similar to the process described above with respect to FIG. 3A, the process illustrated in FIG. 3B includes presenting one or more recommendations to a user (e.g., a surgeon, schedule manager, and the like), receiving a selection of a new date for any affected operations and/or prioritization for affected operations, and checking any accepted recommendation for conflicts. Also similar to the discussion above, any update to the time and/or status of an operation can require a note explaining the update.

As illustrated in illustrated in FIG. 3C, the process for reviewing and/or accepting a recommendation can include iterations between one or more users of the operating room optimization system. For example, a recommendation can prompt a surgeon and/or a schedule manager to consider changes to the system. Then, when one of the users accepts a recommendation with a change to an operating room schedule, the change is sent to the other user(s). If the other user(s) also accept, the change is approved. If the other user(s) do not accept, they are prompted to propose an alternative change, which is then sent to the first user (and any other user(s)) as a new proposed change. The process in FIG. 3C can continue until a change is agreed upon by all users and/or until all users agree to decline the recommendation. In some implementations, each approved change and/or non-approved change requires a note indicating a reason for the approval and/or the non-approval, in addition to an update to the schedule.

In some implementations, as discussed above, the recommendation can include a suggestion to schedule one or more additional operations in open time in the operating room. FIG. 3D illustrates a process for requesting additional operation(s) when the recommendation is approved (e.g., accepted). As illustrated, the process begins by receiving one or more procedure requests that indicate relevant and/or necessary information (e.g., the requested time, operating room, patient, procedure type, doctor and/or surgeons involved, and the like) as well as a prioritization. The requested procedures are then sent to a schedule manager for review against conflicts and/or review based on prioritization. When no conflicts are found, the request(s) can be approved and the schedule for the operating room can be updated. Conversely, when a conflict is found, the request(s) can be returned with an indication for the non-approval, and new requests can be submitted.

FIG. 3E illustrates a process for improving the utilization of an operating room in accordance with some implementations of the present technology. As illustrated in FIG. 3E, the process begins by receiving data related to the operating room (e.g., information related to past recommendations (e.g., approvals, rejections, reasons for rejections, and the like), future schedules for the operating room, updates to the schedules, and the like). The data is then aggregated and stored to update any applicable regression models in the system during future runs. Further, any identified updates are placed into batches and sent to relevant recipients (e.g., surgeons, staff schedulers, and the like).

Table 1 below lists several features of the optimized operating room block management system. One of skill in the art will understand that the system can implement zero or more of the features listed in Table 1.

TABLE 1

| Feature Name | Description |
|---|---|
| Single Sign On | Users can be authenticated using their organization's |
| | credentials |
| Returning User Welcome | Non-new users have a message welcoming them back to the |
| | optimized operating room block management system. |
| Calendar: Views | Users can view a chart of Block time and Procedure time in |
| | a Day and Week view. |
| Calendar: Filtering | Scheduler can filter to show/hide specific blocks and |
| | procedures, including: |
| | Downstream/upstream |
| | Service line/Surgical group |
| | Location/OR room |
| | Other surgeons |
| | Type of procedure |
| | Recommended Block releases |
| | Specific Date |
| Calendar: Indicators | There are color indicators for each of the following: |
| | Release time |
| | Recommended |
| | Pending |
| | Released |
| Block Release: Views | Users can select to view New, All, Pending, and Released |
| | Blocks |
| Block Release: Block Details | Users can see the following information in blocks that are |
| | on the schedule: |
| | Date, Time |
| | OR Room, Location |
| | Leave a note (optional) |
| Block Release: Accept | Users can accept a recommendation to release a block. |
| Recommended Block Release | This can generate a confirmation message. |
| Block Release: Reject | Users can accept a recommendation to release a block. |
| Recommended Block Release | This generates a confirmation message. The |
| | recommendation would show as “rejected” in the |
| | “Requested” view for a pre-determined amount of time. |
| Block Release algorithm | |
| Block Release recommendations | |
| Block Release: Manually Release | Users can select a block of time to release. |
| Block | |
| Procedure Relative Complexity | How complex on a scale of 1-5 is this procedure in |
| | comparison to average? |
| Upcoming Schedule Calendar: | The home page of the app shows upcoming surgeries and |
| Home Page Preview | relevant details. |
| Details view of specific procedure | Users can view details of an upcoming schedule item. |
| Upcoming Schedule Calendar: | Users can see a calendar view of their upcoming schedule, |
| View | including filtering and view change. |
| | Users can swipe to slide to previous/next |
| Upcoming Schedule Calendar: | Schedulers can filter to show/hide specific pieces of |
| Filters | information, including what is upstream/downstream, the |
| | service line/surgical group, the location/OR room, and the |
| | type of procedure. |
| Notification: Suggested Block | Notify users when there are suggested blocks to release. |
| Release | |
| Notification: See All | Users can see active and recent notifications. |
| Notifications: Status updates from | When a status change happens on a requested procedure |
| requested procedures | from a scheduler/admin, the user receives a notification with |
| | these options: |
| | Accepted |
| | Needs more Information |
| | Rejected (with note) |
| Ability for partial block release | |
| Upcoming Schedule Calendar: | Users have the ability to link out to electronic medical |
| Link out to one or more electronic | records systems to see additional case information. |
| medical records systems | |
| First Time User: Welcome | Welcome information for new users. |
| First Time User: User Nickname | Gathering a “nickname” for UX-driven greetings. |
| First Time User: Designation | Confirming admin-entered information: role, specialty, |
| Confirmation | facilities - with prompt to contact org admin if anything is |
| | incorrect. |
| Micro-tutorials | 1-2 micro-tutorials for first time users explaining a key |
| | function of the app. |
| Procedure: Edit | Users can edit procedure information. |
| Upcoming Schedule Calendar: | Users have the option to see the previous/next procedures |
| Upstream/downstream | adjacent to their own procedures (e.g., what surgeon are you |
| | waiting for to start your procedure? What surgeon will be |
| | waiting for you to finish?) |
| Procedure Request | Users can mark which requests are “urgent.” Possibly with |
| | some additional impact to deter from selecting this. |
| Procedure Request | Users have the following options if no open procedure time |
| | meets the requested criteria: |
| | Show adjacent free time, with ability to select available |
| | adjacent time. |
| | For user to be notified if something meeting that criteria |
| | becomes available. |
| Notifications: Formats | Users can received notifications in the following formats: |
| | In-app alerts |
| | Text message alerts |
| Notifications: Dismiss Reminders | Reminders that are dismissed generate a reminder after a set |
| | amount of time. |
| Multi-language support | Users can select the language they would like the app to be |
| | displayed in. |
| Release to member of physician | |
| group | |
| Credentialing at multiple facilities - | |
| drive times, patient class, | |
| patient data (e.g., BMI, age, | |
| demographic data, etc.) | |
| Incentivization of case request | |
| parameters | |
| Streamline communication | |
| pathway for ‘going over’ block | |
| time | |
| Case length estimation | Predicted case length. |
| Block release prompts | Prompts will include rationale for block exchange (i.e., |
| | release and time will be filled). |
| Upstream and downstream | |
| procedures viewable | |
| Notifications: Types | A user can receive the following types of notifications: |
| | Updated/expected start times |
| | Probability of exceeding block allocation |
| | Consent confirmations |
| | Block status changes |
| Status legend | Users need to be able to understand what each status, |
| | “New,” “Pending,” “Declined,” “Successful,” and |
| | “Unsuccessful” mean from a workflow standpoint. |
| Procedure Request | Users can select to view All, Pending, or Requested |
| | Procedure Requests. |
| Procedure Request | Users can select a range of items filtered by when |
| | requesting a procedure, including: Date range, Time (any or |
| | specific), Type of procedure, Location (any or multiple), OR |
| | Room #, Duration, Leave a note (optional). |
| Procedure Request | 1. Pending 2. Success 3. Rejected (if the available time |
| | option is expired). |
| Procedure Request: Multiple | Ability to put in multiple procedure codes. |
| codes | |
| Common procedure list | Have ‘most likely’ procedure dropdown - first 10 procedures |
| | in drop-down list are the historically most frequent for the |
| | surgeon (group admins can schedule a procedure for a |
| | surgeon). |
| Links to electronic medical | Links that allows surgeons to see procedures in electronic |
| records systems | medical records systems or related apps where procedure |
| | details are displayed (homepage and schedule [proc popup] |
| | link to see that specific procedure). |
| Messaging systems | Messaging systems for automatic data pulls and write back |
| | access. |
| Integration with email/calendar | |
| systems | |
| Block Release: Block Details | Probability that time will go unused based on past booking |
| Advanced | patterns. |
| | Probability that time will be picked up by another surgeon |
| | if released now. |
| Select metrics | |
| First Time User: Create password | First time users that do not have Single Sign On (SSO) |
| | create a password as part of their setup. |
| Ability to edit procedures, notify | |
| OR Scheduler | |
| OR Scheduler to edit procedures, | |
| notify surgeon | |

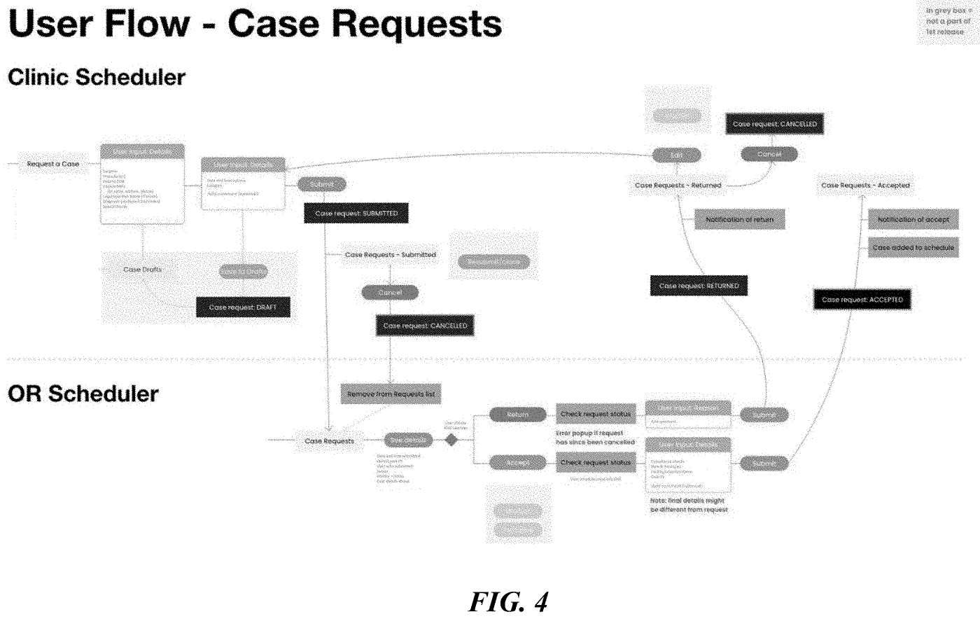

FIG. 4 is a flow diagram illustrating a process performed by some implementations of an optimized operating room block management system to handle case requests. As illustrated in FIG. 4, the process can begin with a request for a case (e.g., a request for an operation, a request for access to the operating room, and the like). The request includes details such as the surgeon for the operation, the procedure, details about the patient's background and identification, diagnosis information, special needs and/or requests, and the like. The request also includes an indication of a preferred date, time, and location. In some implementations, case drafts can be created, saved, updated, then submitted into the process. For example, a case draft can be created when a patient first seeks treated, updated as diagnosis information is received, then submitted when a procedure is selected. Any time after a case request is submitted, it can be canceled by a suitable party and the cancelation can be automatically sent to all relevant parties.

Once a case request is submitted, the request is sent to an operating room scheduler. The operating room scheduler can review the request, a current schedule for the operating room, any relevant recommendations for updating the schedule (e.g., a recommendation that an interval in a block date will be available for an operation), and/or any related information on available resources (e.g., staff schedules). The operating room scheduler can the approve or return the case request. And approved request is entered into the system and an indication of the approval is sent to the requesting users. A returned request is sent back to the requesting user, who then has an opportunity to update the request in view of any included information (e.g., the recommendation for the operating room indicates that no additional operations should be scheduled for a given day, that the request should be for an alternative time in a given day, that a conflict is present preventing scheduling at a certain time, and the like). The user can then waitlist the request in case the schedule is later updated and/or edit their request for another time.

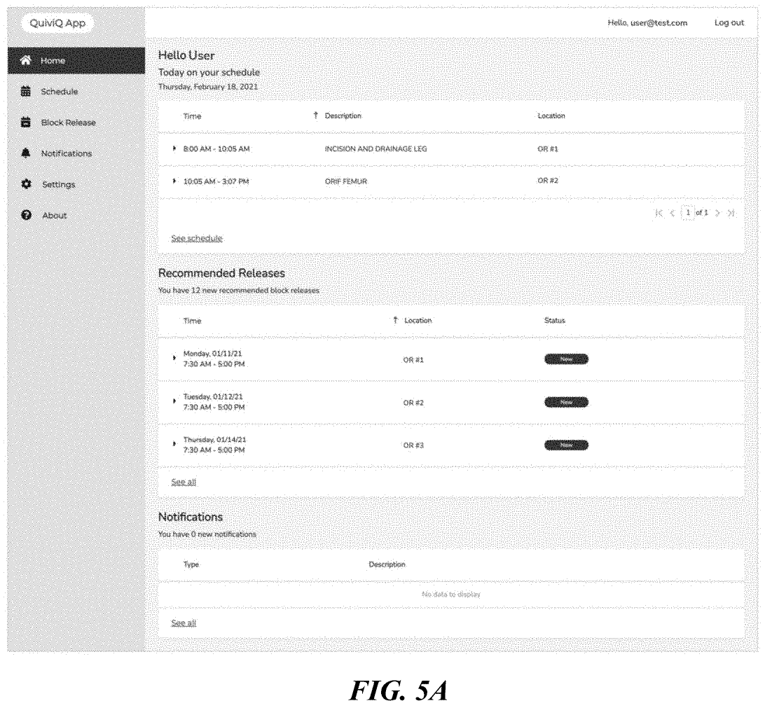

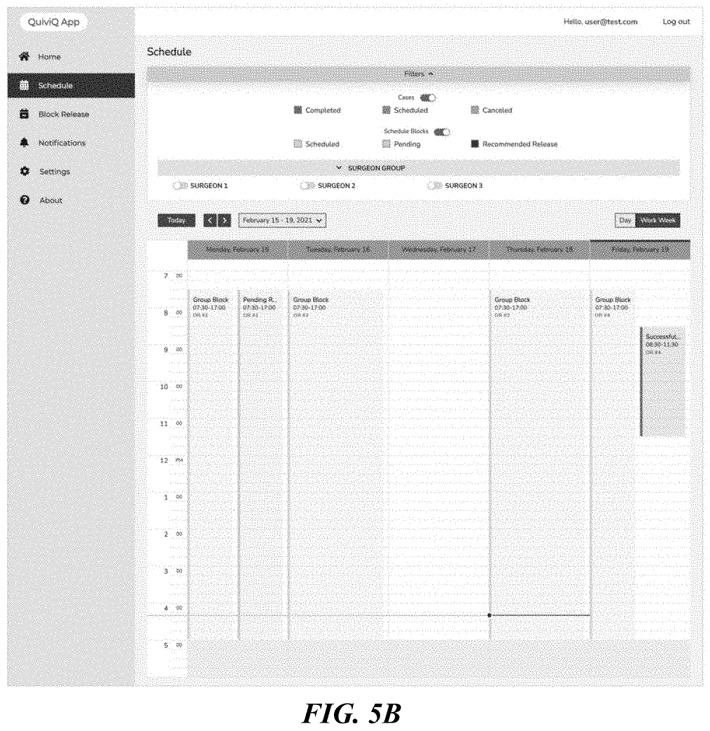

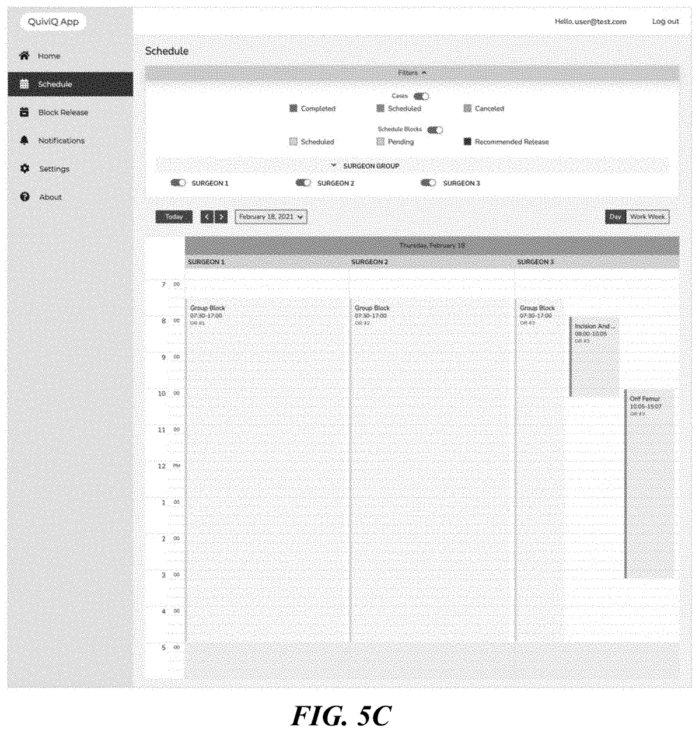

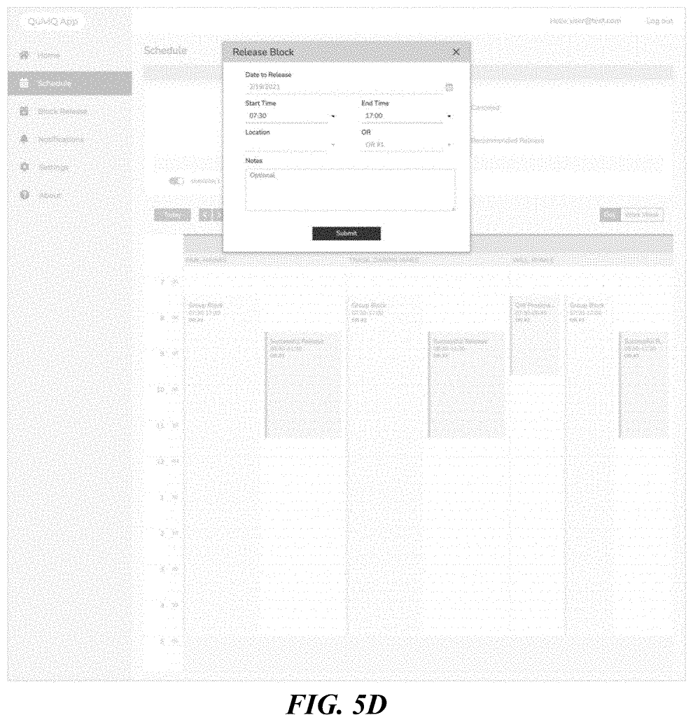

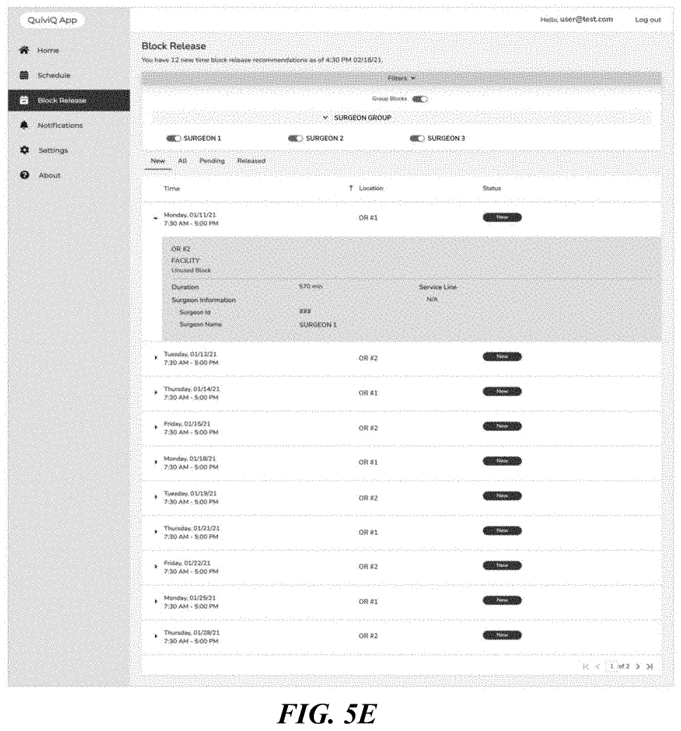

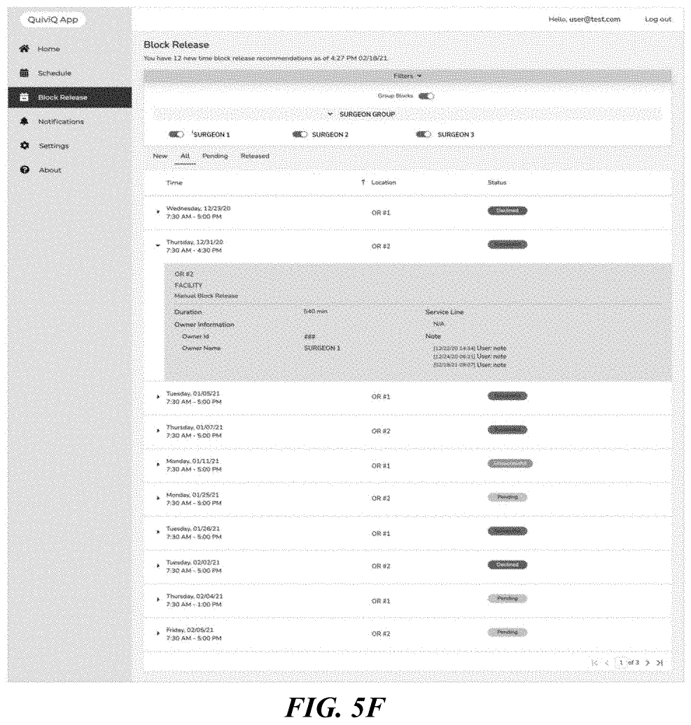

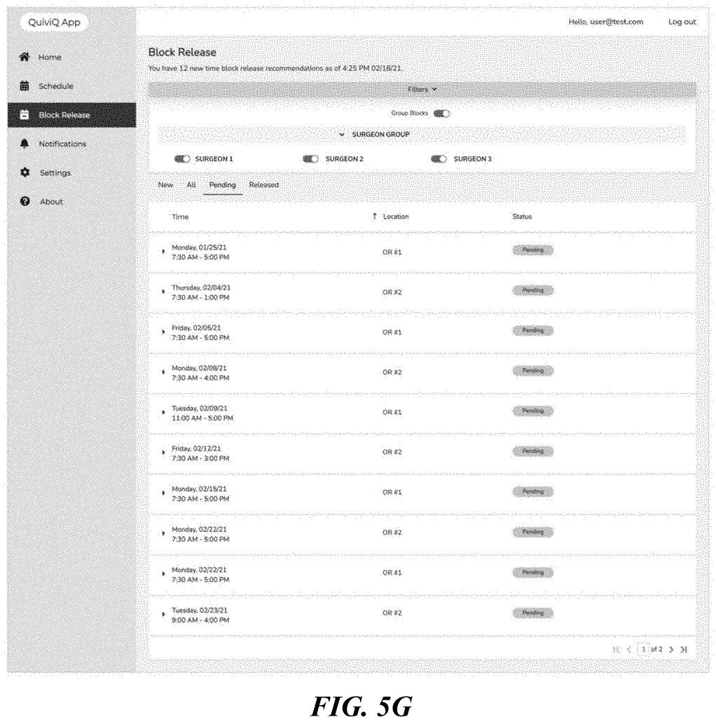

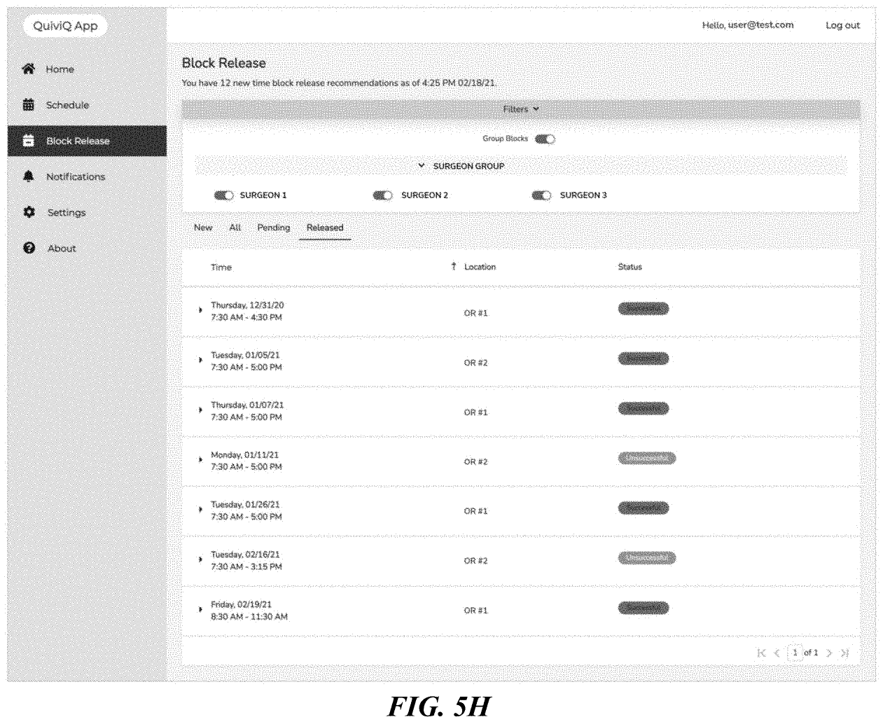

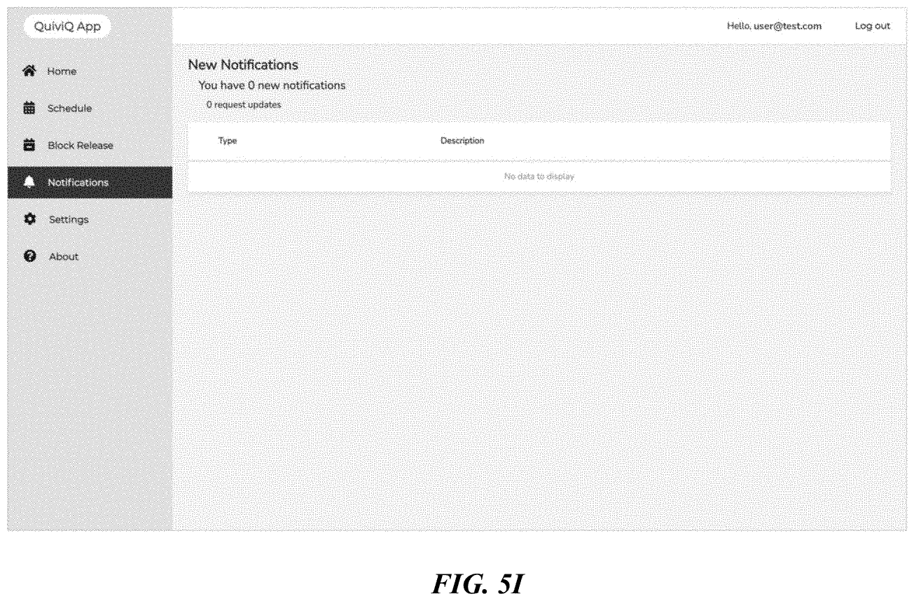

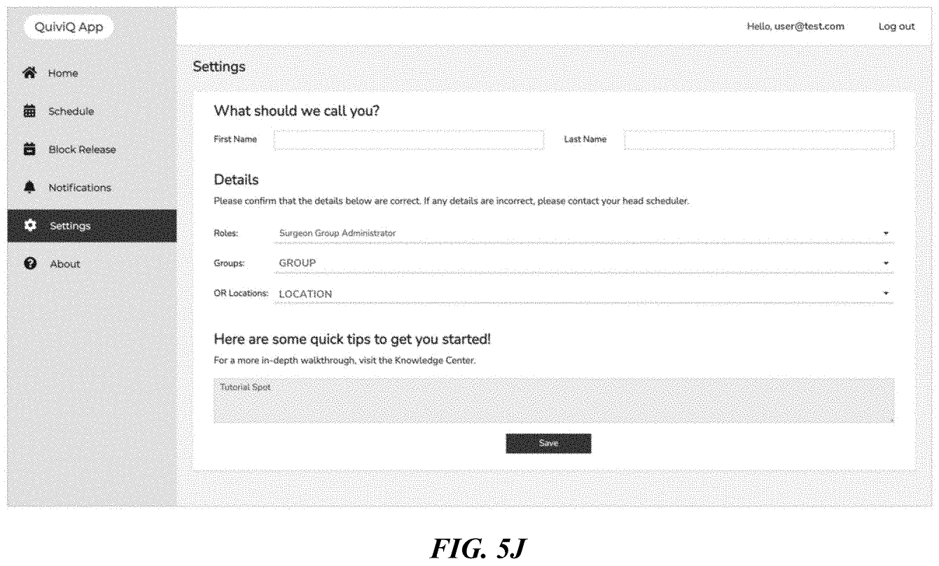

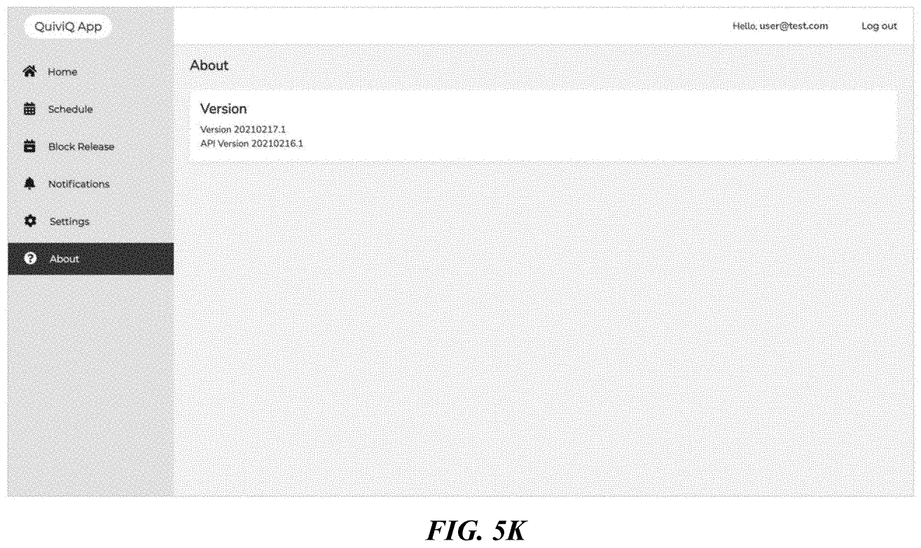

FIGS. 5A-5K are example user interfaces utilized by some implementations of an optimized operating room block management system for operating room schedule management.

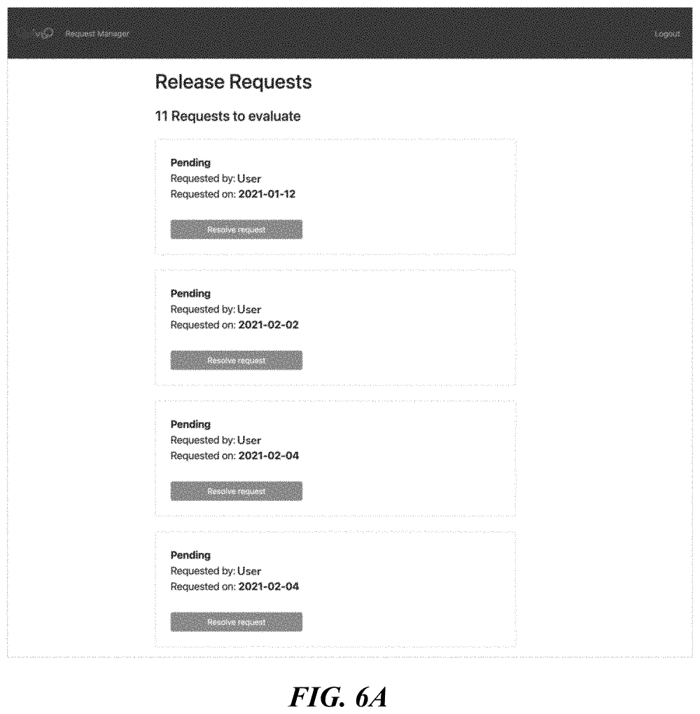

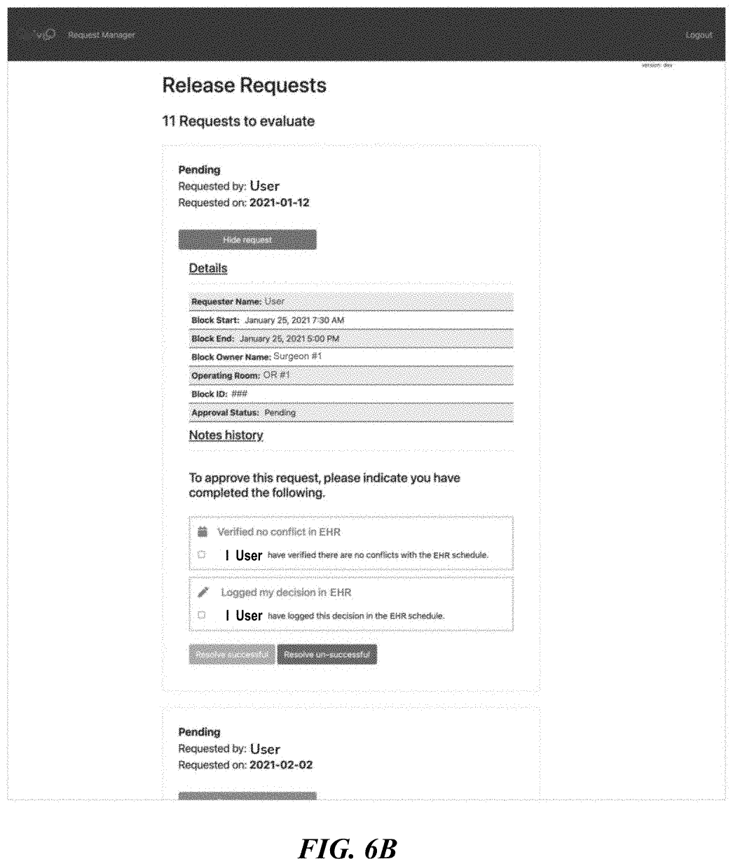

FIGS. 6A-6B are example user interfaces utilized by some implementations of an optimized operating room block management system for operating room request management.

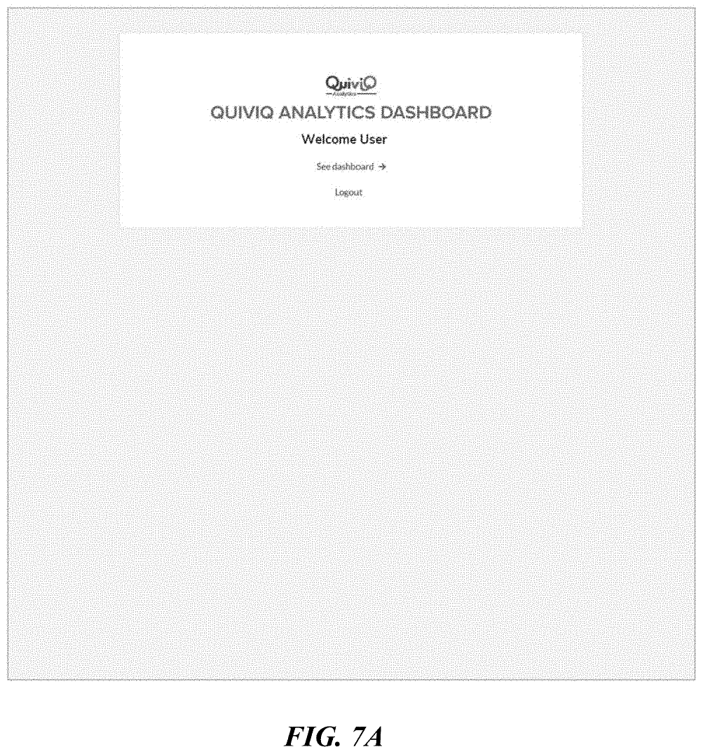

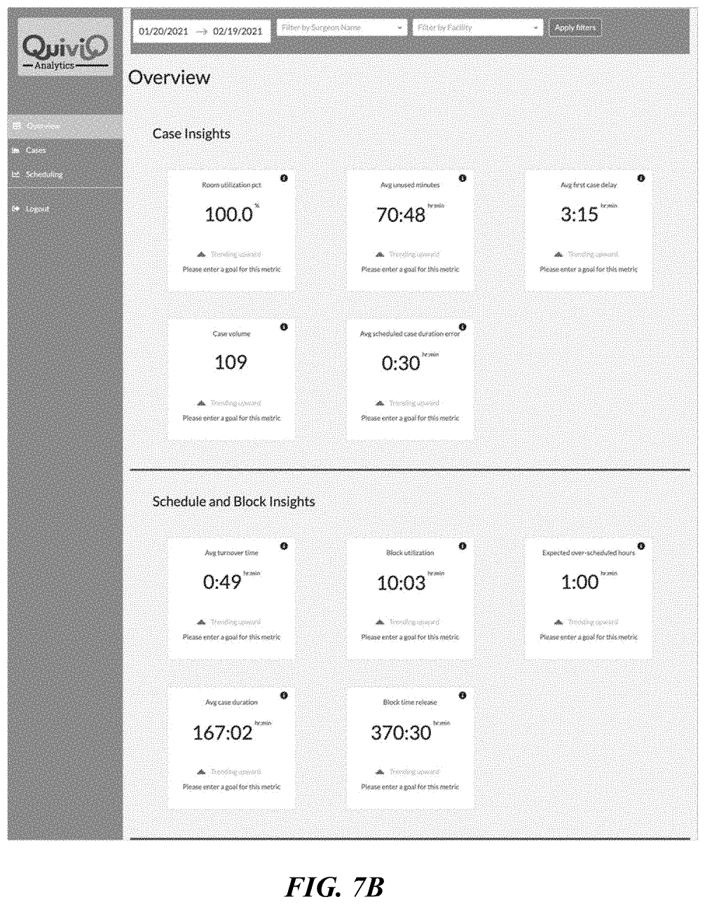

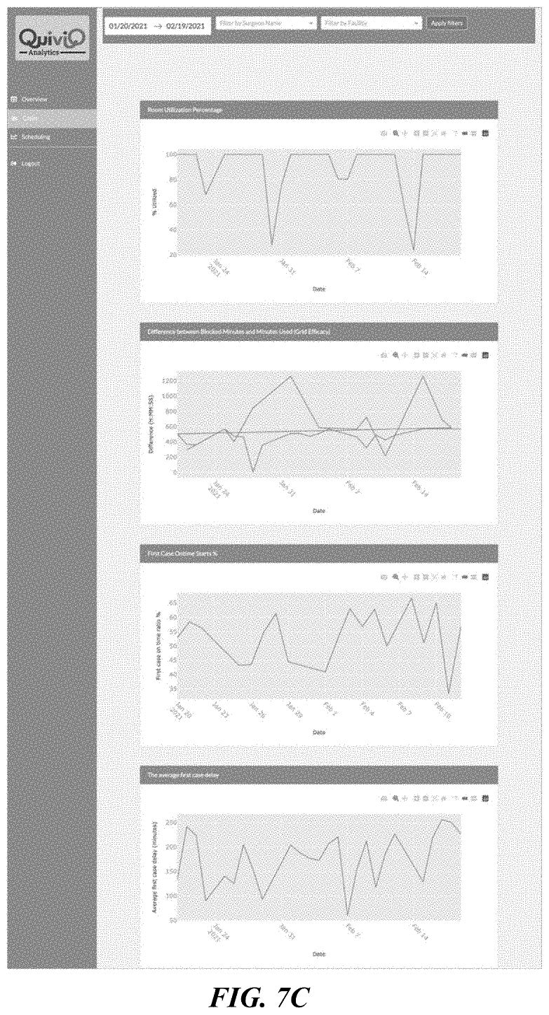

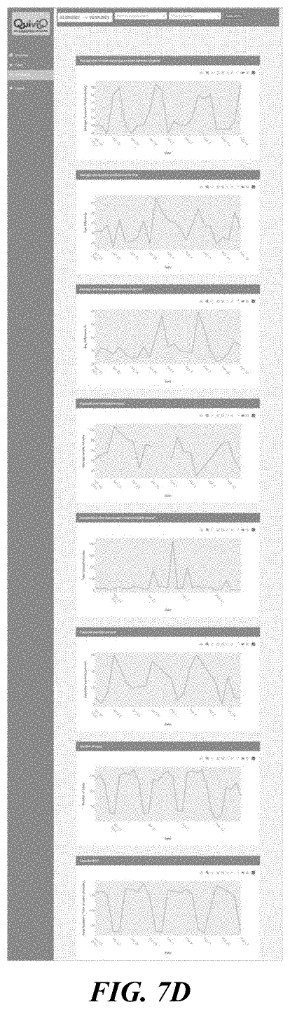

FIGS. 7A-7D are example user interfaces utilized by some implementations of an optimized operating room block management system for performing analytics on the operating room data. The optimized operating room block management system can compute and present metrics at various granularities, such as daily, weekly, monthly, quarterly, and annually. Table 2 provides some example metrics generated and/or provided by the optimized operating room block management system.

TABLE 2

| Objective | Metric | Description | Formula |
|---|---|---|---|
| Turnover Time | Average | Mean downtime (min) | E[min(60 minutes, Start time of |
| | Turnover Time | between surgeries (time | Procedure N + 1 − End time of |
| | | to clean and prepare | Procedure N)] |
| | | room) | |
| Capacity | Difference | Difference (min) | Expected block minutes − |
| Management | between | between expected Block | Actual block minutes used. |
| | Blocked Minutes | grid and actuals | Actual = sum of In Room |
| | and Minutes | | duration + sum of turnover time |
| | Used (Grid | | (all logged cases that occurred |
| | Efficacy) | | with any primary surgeon |
| | | | associated with the block of |
| | | | interest, regardless of operating |
| | | | room, and hour of the day). |
| | | | Expected = sum of block |
| | | | minutes (Calculate by day of |
| | | | week. Group by Month) |
| | | | Can be broken down by |
| | | | surgeon by group by Day of |
| | | | week, etc. |
| Room | Room | Percent of primetime (%) | (Actual turnover during |
| Utilization | Utilization | with scheduled cases. | primetime + case duration |
| | Percentage | (Only active rooms) | minutes during primetime)/ |
| | | | total minutes of primetime. |
| Scheduled | Average Case | Mean difference (min) | E[abs(actual procedure time − |
| Case Duration | Duration | between scheduled | scheduled procedure time)] |
| Accuracy | Prediction Error | procedure time and | |
| | | actual procedure time | |
| Scheduled | Average % Case | Mean difference (min) | E[abs(actual procedure time − |
| Case Duration | Duration | between scheduled | scheduled procedure |
| Accuracy | Prediction Error | procedure time and | time)]/(scheduled procedure |
| | | actual procedure time, | time) |
| | | expressed as a percent of | |
| | | scheduled time | |
| Start Time | First Case | Percent (%) of first cases | # times that first starts ontime/ |
| Tardiness | Ontime Starts % | that start on time | total number of first cases |
| Start Time | Expected First | Mean delay (min) of first | E[max(0, first case actual start |
| Tardiness | Case Delay | case | time − first case scheduled start |
| | | | time)] |
| Capturable | Unused block | Sum (min) of unused | turnover time in excess of 60 |
| Time | time that | block time greater than | minutes (and % of turnovers |
| | exceeds | 60 contiguous minutes | that exceed 60 minutes) |
| | minimum usable | | |
| | amount | | |
| Excess Staffing | Expected Over- | Mean overtime (min) | E[max(0, (Actual End Time − |
| Costs | scheduled Hours | required to complete all | Scheduled End Time)] |
| | | of a block's scheduled | |
| | | procedures (time past the | |
| | | block end time) | |
| Number of | Number of | Count of cases (#) | Trended by procedure by |
| Cases | Cases | | grouping level (surgeon, group, |
| | | | Service Line, Procedure, OR) |
| Case Duration | Case Duration | Sum (min) of total in | Trended by procedure by |
| by Procedure | | room procedure time. | grouping level (surgeon, group, |
| | | Comparable across | OR) |
| | | Surgeons and Facilities | |
| Scheduling | Block Time | Sum (min) of Block | sum(block release minutes) |
| | Released | Time Released by | |
| | | method of release | |
| | | (notification vs. manual | |
| | | vs. automatic) | |
| Excess Staffing | Expected | % of procedures | E[max(0, (Actual End time − |
| Costs | Overtime % | requiring overtime past | Scheduled Start |
| | | block end | Time)/(Scheduled End Time − |
| | | Number of times a cases | Scheduled Start Time)] |
| | | end time occurs after a | Of cases with a start time inside |
| | | block's end time | a block . . . |
| | | | Over Block = if (Block End |
| | | | Timestamp < Out of Room, 1, 0) |
| | | | Num Blocks = count(Blocks) |
| | | | Over Block/Num Blocks |

In some implementations, the optimized operating room block management system can manage permissions for viewing information related to the schedule user interfaces (FIGS. 5A-5K) based on user roles. For example, a user with a “Surgeon” role can view the following information: all “personal” surgeon procedures (completed, pending, scheduled, NOT canceled), all “personal” surgeon blocks (scheduled, pending), recommended releases, group blocks for which this surgeon is a member (when there are no procedures already scheduled into that block). The user with a “Surgeon” role can edit the following information: scheduled procedures (all fields), scheduled “personal” surgeon blocks (only the time fields in order to release a partial block), pending procedures (all fields), pending “personal” surgeon blocks (only the time fields), and so on. The user with a “Surgeon” role cannot edit the following information: completed procedures, cancelled procedures, recommended releases, and group blocks. The user with a “Surgeon” role can release the following information: scheduled “personal” surgeon blocks (either by releasing the entire block or editing time fields to release partial), and recommended releases (entire block) and can also decline these. The user with a “Surgeon” role cannot release group blocks, can request procedure, and can cancel (delete) procedure. A user with a “Group Admin” role can view all surgeon procedures of surgeons within their group, all surgeon blocks of surgeons within their group, and group blocks.

In some implementations, the optimized operating room block management system can integrate with one or more email service clients (e.g., Outlook, Gmail, etc.). For example, one or more of the schedule user interfaces (FIGS. 5A-5K) can present an integrated view with a user's email service clients. The schedule interfaces can display data at various granularities, such as day, work week, full week, month, year, and so on. The schedule interfaces can display one or more of the following information regarding procedures: patient name, date of birth, procedure type, facility, operating room, expected procedure length, surgical assistant(s), and so on. The schedule interfaces can display one or more of the following data points regarding blocks: facility, operating room, time bounds, physician name, and so on. The schedule interfaces can display work queue and schedule in a single view to schedulers.

### III. Examples of Suitable Computer Systems

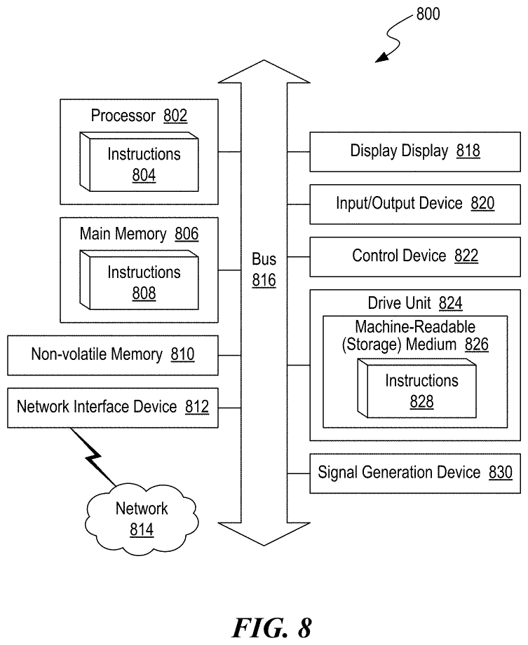

FIG. 8 is a block diagram that illustrates an example of a computer system 800 in which at least some operations described herein can be implemented. As shown, the computer system 800 can include one or more processors 802, main memory 806, non-volatile memory 810, a network interface device 812, video display device 818, an input/output device 820, a control device 822 (e.g., keyboard and point device), a drive unit 824 that includes a storage medium 826, and a signal generation device 830 that are communicatively connected to a bus 816. The bus 816 represents one or more physical buses and/or point-to-point connections that are connected by appropriate bridges, adapters, or controllers. The bus 816 therefore can include a system bus, a Peripheral Component Interconnect (PCI) bus or PCI-Express bus, a HyperTransport or industry standard architecture (ISA) bus, a small computer system interface (SCSI) bus, a universal serial bus (USB), IIC (I2C) bus, or an IEEE standard 1394 bus (also referred to as “Firewire”). Various common components (e.g., cache memory) are omitted from FIG. 8 for brevity. Instead, the computer system 800 is intended to illustrate a hardware device on which components illustrated or described relative to the examples of the figures and any other components described in this specification can be implemented.

The computer system 800 can take any suitable physical form. For example, the computing system 800 can share a similar architecture as that of a personal computer (PC), tablet computer, mobile telephone, game console, music player, wearable electronic device, network-connected (“smart”) device (e.g., a television or home assistant device), AR/VR systems (e.g., head-mounted display), or any electronic device capable of executing a set of instructions that specify action(s) to be taken by the computing system 800. In some implementation, the computer system 800 can be an embedded computer system, a system-on-chip (SOC), a single-board computer system (SBC) or a distributed system, such as a mesh of computer systems, or include one or more cloud components in one or more networks. Where appropriate, one or more computer systems 800 can perform operations in real-time, near real-time, or in batch mode.

The processor 802 can be, for example, a central processing unit or a conventional microprocessor (e.g., Intel Pentium processor). The memory (e.g., main memory 806, non-volatile memory 810, machine-readable medium 826) can be local, remote, or distributed. Although shown as a single medium, the machine-readable medium 826 can include multiple media (e.g., a centralized/distributed database and/or associated caches and servers) that store one or more sets of instructions 828. The machine-readable (storage) medium 826 can include any medium that is capable of storing, encoding, or carrying a set of instructions for execution by the computing system 800. One of skill in the relevant art will recognize that the machine-readable medium 826 can include any type of medium that is accessible by the processor. The machine-readable medium 826 can be non-transitory or comprise a non-transitory device. In this context, a non-transitory storage medium can include a device that is tangible, meaning that the device has a concrete physical form, although the device can change its physical state. Thus, for example, non-transitory refers to a device remaining tangible despite this change in state.

In general, the routines executed to implement the implementations of the disclosure can be implemented as part of an operating system or a specific application, component, program, object, module, or sequence of instructions (collectively referred to as “computer programs”). The computer programs typically comprise one or more instructions (e.g., instructions 804, 808, 828) set at various times in various memory and storage devices in computing device(s). When read and executed by the processor 802, the instruction(s) cause the computing system 800 to perform operations to execute elements involving the various aspects of the disclosure.

Although implementations have been described in the context of fully functioning computing devices, the various examples are capable of being distributed as a program product in a variety of forms. Examples of machine-readable storage media, machine-readable media, or computer-readable media include recordable-type media, such as volatile and non-volatile memory devices 810, removable flash memory, hard disk drives, optical disks, and transmission-type media, such as digital and analog communication links.

Software is typically stored in the non-volatile memory and/or the drive unit 824. When software is moved to the memory for execution, the processor 802 will typically make use of hardware registers to store values associated with the software and local cache that can serve to speed up execution. As used herein, a software program is assumed to be stored at any known or convenient location (e.g., non-volatile storage, hardware registers) when the software program is referred to as “implemented in a computer-readable medium.” A processor can be “configured to execute a program” when at least one value associated with the program is stored in a register readable by the processor.

The network interface device 812 enables the computing system 800 to mediate data in a network 814 with an entity that is external to the computing system 800 through any communication protocol supported by the computing system 800 and the external entity. Examples of the network interface device 812 include a network adaptor card, a wireless network interface card, a router, an access point, a wireless router, a switch, a multilayer switch, a protocol converter, a gateway, a bridge, a bridge router, a hub, a digital media receiver, and/or a repeater.

Further, the interface device 812 can include a firewall that governs and/or manages permission to access/proxy data in a computer network and tracks varying levels of trust between different machines and/or applications. The firewall can be any number of modules having any combination of hardware and/or software components able to enforce a predetermined set of access rights between a particular set of machines and applications, machines and machines, and/or applications and applications (e.g., to regulate the flow of traffic and resource sharing between these entities). The firewall can additionally manage and/or have access to an access control list that details permissions including the access and operation rights of an object by an individual, a machine, and/or an application, and the circumstances under which the permission rights stand.

Examples of the I/O devices 820 include a keyboard, a mouse or other pointing device, disk drives, printers, a scanner, and other input and/or output devices, including a display device. Examples of the display device 818 can include a cathode ray tube (CRT), liquid crystal display (LCD), or any display device.

In operation, the computer system 800 can be controlled by operating system software that includes a file management system, such as a disk operating system. One example of operating system software with associated item management system software is the family of operating systems known as Windows® from Microsoft Corporation of Redmond, Wash., and their associated item management systems. Another example of operating system software with its associated item management system software is the Linux™ operating system and its associated item management system. The item management system is typically stored in the non-volatile memory and/or drive unit and causes the processor to execute the various acts required by the operating system to input and output data and to store data in the memory, including storing items on the non-volatile memory and/or drive unit.

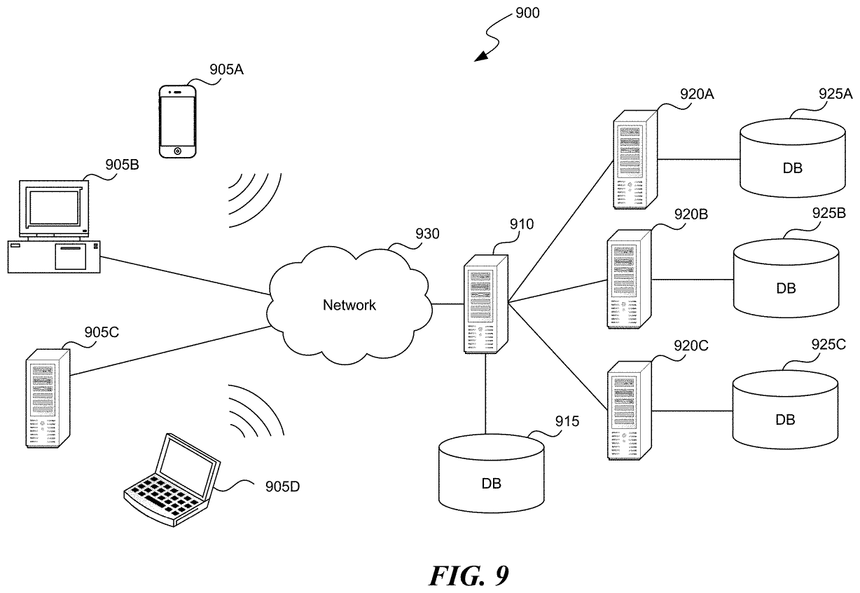

FIG. 9 is a system diagram illustrating an example of a computing environment in which the disclosed system operates in some implementations. In some implementations, environment 900 includes one or more client computing devices 905A-D, examples of which can host the system 100. Client computing devices 905 operate in a networked environment using logical connections through network 930 to one or more remote computers, such as a server computing device.

In some implementations, server 910 is an edge server which receives client requests and coordinates fulfillment of those requests through other servers, such as servers 920A-C. In some implementations, server computing devices 910 and 920 comprise computing systems, such as the system 100. Though each server computing device 910 and 920 is displayed logically as a single server, server computing devices can each be a distributed computing environment encompassing multiple computing devices located at the same or at geographically disparate physical locations. In some implementations, each server 920 corresponds to a group of servers.

Client computing devices 905 and server computing devices 910 and 920 can each act as a server or client to other server or client devices. In some implementations, servers (910, 920A-C) connect to a corresponding database (915, 925A-C). As discussed above, each server 920 can correspond to a group of servers, and each of these servers can share a database or can have its own database. Databases 915 and 925 warehouse (e.g., store) information such as a list of donors, information about a political committee, vendor information, political finance rules, financial data, and so on. Though databases 915 and 925 are displayed logically as single units, databases 915 and 925 can each be a distributed computing environment encompassing multiple computing devices, can be located within their corresponding server, or can be located at the same or at geographically disparate physical locations.

Network 930 can be a local area network (LAN) or a wide area network (WAN), but can also be other wired or wireless networks. In some implementations, network 930 is the Internet or some other public or private network. Client computing devices 905 are connected to network 930 through a network interface, such as by wired or wireless communication. While the connections between server 910 and servers 920 are shown as separate connections, these connections can be any kind of local, wide area, wired, or wireless network, including network 930 or a separate public or private network.

The technologies introduced here can be implemented by programmable circuitry (e.g., one or more microprocessors), software and/or firmware, special-purpose hardwired (e.g., non-programmable) circuitry, or a combination of such forms. Special-purpose circuitry can be in the form of one or more application-specific integrated circuits (ASICs), programmable logic devices (PLDs), field-programmable gate arrays (FPGAs), etc.

Some portions of the disclosure can be presented in terms of algorithms and symbolic representations of operations on data bits within a computer memory. These algorithmic descriptions and representations are the means used by those skilled in the data processing arts to most effectively convey the substance of their work to others skilled in the art. An algorithm can refer to a sequence of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing,” “computing,” “calculating,” “determining,” “displaying,” “generating,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission, or display devices.

The algorithms and displays presented herein are not inherently related to any particular computer or other apparatus. Various general-purpose systems can be used with programs in accordance with the described teachings, or it can prove convenient to construct more specialized apparatus to perform the methods of some implementations. The required structure for a variety of these systems will appear from the description. In addition, the techniques are not described with reference to any particular programming language, and various implementations can thus be implemented using a variety of programming languages.

In some circumstances, operation of a memory device, such as a change in state from a binary one to a binary zero or vice-versa, for example, can comprise a transformation, such as a physical transformation. With particular types of memory devices, such a physical transformation can comprise a physical transformation of an article to a different state or thing. For example, but without limitation, for some types of memory devices, a change in state can involve an accumulation and storage of charge or a release of stored charge. Likewise, in other memory devices, a change of state can comprise a physical change or transformation in magnetic orientation or a physical change or transformation in molecular structure, such as from crystalline to amorphous or vice versa. The foregoing is not intended to be an exhaustive list in which a change in state from a binary one to a binary zero or vice-versa in a memory device can comprise a transformation, such as a physical transformation. Rather, the foregoing is intended as illustrative examples.

### Remarks

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof means any connection or coupling, either direct or indirect, between two or more elements; the coupling or connection between the elements can be physical, logical, or a combination thereof. Additionally, the words “herein,” “above,” “below,” and words of similar import can refer to this application as a whole and not to any particular portions of this application. Where context permits, words in the above Detailed Description using the singular or plural number may also include the plural or singular number, respectively. The word “or” in reference to a list of two or more items covers all of the following interpretations of the word: any of the items in the list, all of the items in the list, and any combination of the items in the list. The term “module” refers broadly to software components, firmware components, and/or hardware components.

While specific examples of technology are described above for illustrative purposes, various equivalent modifications are possible within the scope of the invention, as those skilled in the relevant art will recognize. For example, while processes or blocks are presented in a given order, alternative implementations can perform routines having steps, or employ systems having blocks, in a different order, and some processes or blocks may be deleted, moved, added, subdivided, combined, and/or modified to provide alternative or sub-combinations. Each of these processes or blocks can be implemented in a variety of different ways. Also, while processes or blocks are at times shown as being performed in series, these processes or blocks can instead be performed or implemented in parallel, or can be performed at different times. Further, any specific numbers noted herein are only examples such that alternative implementations can employ differing values or ranges.

Details of the disclosed implementations can vary considerably in specific implementations while still being encompassed by the disclosed teachings. As noted above, particular terminology used when describing features or aspects of the invention should not be taken to imply that the terminology is being redefined herein to be restricted to any specific characteristics, features, or aspects of the invention with which that terminology is associated. In general, the terms used in the following claims should not be construed to limit the invention to the specific examples disclosed herein, unless the above Detailed Description explicitly defines such terms. Accordingly, the actual scope of the invention encompasses not only the disclosed examples, but also all equivalent ways of practicing or implementing the invention under the claims. Some alternative implementations can include additional elements to those implementations described above or include fewer elements.

Any patents and applications and other references noted above, and any that may be listed in accompanying filing papers, are incorporated herein by reference in their entireties, except for any subject matter disclaimers or disavowals, and except to the extent that the incorporated material is inconsistent with the express disclosure herein, in which case the language in this disclosure controls. Aspects of the invention can be modified to employ the systems, functions, and concepts of the various references described above to provide yet further implementations of the invention.

To reduce the number of claims, certain implementations are presented below in certain claim forms, but the applicant contemplates various aspects of an invention in other forms. For example, aspects of a claim can be recited in a means-plus-function form or in other forms, such as being embodied in a computer-readable medium. A claim intended to be interpreted as a mean-plus-function claim will use the words “means for.” However, the use of the term “for” in any other context is not intended to invoke a similar interpretation. The applicant reserves the right to pursue such additional claim forms in either this application or in a continuing application.

### Examples

The present technology is illustrated, for example, according to various aspects described below. These are provided as examples and do not limit the present technology. It is noted that the implementations of the examples below can be combined in any suitable manner, and placed into another described example.

In one example, a method for improving operating room utilization includes retrieving statistics indicative of usage rates for an operating room. The statistics can include, for each individual previous time block in a plurality of previous time blocks: a planned usage rate of the operating room for the individual previous time block; an actual usage rate of the operating room for the individual previous time block; and/or metadata indicating a reason for differences between the planned usage rate and the actual usage rate. The method can also include predicting an estimate of unused time for a future time block. The prediction process can include constructing one or more regression models based on the statistics indicative of usage rates for the operating room, where each of the regression model(s) identifies a relationship between the planned usage rate and the actual usage rate. The prediction process can then include identifying a best fit model from the regression model(s), and applying the best fit model to planned future usage rates for the operating room. Once the estimate is obtained, the method can also include generating a recommendation for the unused time in the estimate. In this example, the recommendation can be based at least partially on an identification of one or more intervals in the future time block associated with the operating room being open in the estimate. In some examples, the recommendation can additionally, or alternatively, be based at least partially on a predetermined amount of required open time (e.g., to accommodate emergency procedures).

In various examples, the recommendation can include: releasing one or more of the identified intervals for additional procedures in the operation room; rescheduling one or more procedures during the future block into one of the identified intervals to increase an amount of continuously available time during the future block and/or to reduce down time for operating room personnel during the future block; closing the operating room during one or more of the identified intervals; and/or various other suitable alternations to the operating room schedule.

In various examples, the one or more regression models can include a Poisson model, a Negative Binomial model, a Bayesian model, a Lasso model, a Polynomial model, and/or various other suitable regression models.

In some examples, generating the recommendation includes determining an amount of available time in each of the identified intervals. When the amount of available time is above a predetermined minimum threshold, the recommendation can include releasing the identified interval for booking for another procedure. Conversely, when the amount of available time is below the predetermined minimum threshold, the recommendation can include closing the operating room during the identified interval and/or releasing personnel assigned to the operating room during the interval (e.g., allowing nurses to be scheduled in another operating room and/or take a scheduled break). In some examples, generating the recommendation includes identifying adjacent intervals in the identified intervals and combining the time in adjacent intervals. Accordingly, the amount of available time in combined intervals can be increased, which can allow the method to identify additional intervals that can be released for another procedure. In some examples, determining the amount of available time includes determining the smaller of an amount of available time identified by the estimate and a maximum time for the interval.

In some examples, generating the recommendation includes determining whether any of the identified intervals is the first interval of the future block (e.g., the beginning of the future time block). When one of the identified intervals is the first interval in the block, generating the recommendation can include determining an amount of time available during the first interval. When the amount of available time is above a predetermined threshold, the recommendation can include releasing the first interval to allow another procedure to be booked. Conversely, when the amount of available time is below the predetermined threshold, the recommendation includes closing the operating room during the first interval. In some examples, recommendations are interdependent recommendations generated for a plurality of operating rooms. For example, when an interval is identified as available for a procedure in a first operating room, a procedure from a second operating room can be recommended to be moved to the first operating room to reduce operating costs of the second operating room, free additional time in the second operating room for more substantial (e.g., longer) procedures, and/or to capitalize on efficient resource deployment between the first and second operating rooms.

In some examples, generating the recommendation includes determining whether any of the identified intervals is a last interval of the future time block (e.g., the end of the future time block). When one of the identified intervals is the last interval, generating the recommendation can include determining an amount of time available during the last interval. In some implementations, determining the amount of time available during the last interval includes: (1) identifying the predicted ending time for the last planned procedure in the future block, (2) identifying the available time between the last planned procedure and the last interval (e.g., the unused time at the end of the interval for the planned procedure), and (3) adding at least a portion of the time difference to the last interval to increase the amount of available time. That is, determining the amount of time available during the last interval can include expanding the last interval into unused time in a previous interval to increase the amount of available time. When the amount of available time is above a predetermined threshold, the recommendation can include releasing the last interval to allow another procedure to be booked. When the amount of available time is below the predetermined threshold, the recommendation can include closing the operating room during the last interval.

In some examples, the method includes send the recommendation to at least of a surgeon, a scheduling manager, an administrator associated with the operating room, a block owner, and a patient for review. In some such examples, the method includes displaying the recommendation to the recipient (e.g., a user of the system related to the method). The method can also include receiving an indication from the user of an approval or a denial of the recommendation. When the recommendation is approved, the method can include updating the planned usage rates during the future block based on the recommendation. When the recommendation is denied, the method can include generating a new recommendation for the unused time in the estimate. In some such examples, the denial includes an indication of one or more reasons for denying the recommendation. Additionally, or alternatively, any new recommendation can be based at least partially on the reason(s) for denying the recommendation

In some examples, the estimate of unused time includes a plurality of sub-estimates each associated with a different level of abstraction for usage of the operating room (e.g., usage rates by medical personnel such as surgeons, doctors, anesthesiologists, radiologists, and the like; usage rates by all operating room staff, such as nurses, pre-operation personnel, post-operation personnel, and the like; and/or usage rates across all facility personnel). Additionally, or alternatively, the recommendation based on the unused time can include recommendations at a variety of levels of abstraction. In some implementations, the recommendation is further based at least partially on a predetermined required amount of unused time for the operating room.

In some examples, the method includes receiving, from a first user (e.g., a patient and/or a relevant medical practitioner), a request for an operating room procedure during the future block. The generated recommendation can then be based at least partially on the request, which can be communicated to a second user. In some examples, the recommendation includes an indication of which of the one or more identified intervals are available to satisfy the at least one request. When the request is either approved or denied, the indication can then be communicated back to the first user.

Another example of the present technology is a computing system having one or more processors and one or more computer-readable mediums storing instructions that, when executed by the processor cause the computing system to perform any of the methods discussed above with any of the limitations on the method discussed above.

### CONCLUSION

From the foregoing, it will be appreciated that specific implementations of the technology have been described herein for purposes of illustration, but well-known structures and functions have not been shown or described in detail to avoid unnecessarily obscuring the description of the implementations of the technology. To the extent any material incorporated herein by reference conflicts with the present disclosure, the present disclosure controls. Where the context permits, singular or plural terms may also include the plural or singular term, respectively. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Furthermore, as used herein, the phrase “and/or” as in “A and/or B” refers to A alone, B alone, and both A and B. Additionally, the terms “comprising,” “including,” “having,” and “with” are used throughout to mean including at least the recited feature(s) such that any greater number of the same features and/or additional types of other features are not precluded. Further, the terms “approximately” and “about” are used herein to mean within at least within 10 percent of a given value or limit. Purely by way of example, an approximate ratio means within a ten percent of the given ratio.

From the foregoing, it will also be appreciated that various modifications may be made without deviating from the disclosure or the technology. For example, one of ordinary skill in the art will understand that various components of the technology can be further divided into subcomponents, or that various components and functions of the technology may be combined and integrated. In addition, certain aspects of the technology described in the context of particular implementations may also be combined or eliminated in other implementations.

Furthermore, although advantages associated with certain implementations of the technology have been described in the context of those implementations, other implementations may also exhibit such advantages, and not all implementations need necessarily exhibit such advantages to fall within the scope of the technology. Accordingly, the disclosure and associated technology can encompass other implementations not expressly shown or described herein.

We claim:

1. A method for improving operating room utilization, the method comprising:
retrieving statistics indicative of usage rates for an operating room, wherein the statistics include, for each individual previous time block in a plurality of previous time blocks:
a planned usage rate of the operating room for the individual previous time block;
an actual usage rate of the operating room for the individual previous time block; and
metadata indicating a reason for differences between the planned usage rate and the actual usage rate;
predicting an estimate of unused time for a future time block, wherein the predicting the estimate includes:
constructing one or more regression models based on the statistics indicative of usage rates for the operating room, wherein each of the one or more regression models identifies a relationship between the planned usage rate and the actual usage rate;
identifying, from the one or more regression models, a best fit model for the statistics indicative of usage rates for the operating room; and
applying the best fit model to planned future usage rates for the operating room during the future time block; and
generating a recommendation for the unused time in the estimate, wherein the recommendation is based at least partially on an identification of one or more intervals in the future time block associated with the operating room being open in the estimate.

2. The method of claim 1 wherein generating the recommendation includes determining, for each of the identified intervals, an amount of available time in the identified interval, and wherein:
when the amount of available time is above a predetermined minimum threshold, the recommendation includes releasing the identified interval for booking, or
when the amount of available time is below the predetermined minimum threshold, the recommendation includes closing the operating room during the identified interval.

3. The method of claim 2 wherein generating the recommendation includes, before determining the amount of available time for each of the identified intervals:
identifying adjacent intervals in the identified intervals; and
combining the adjacent intervals.

4. The method of claim 1 wherein generating the recommendation includes determining, for each of the identified intervals, an amount of available time in the interval, and wherein the amount of available time is equal to the smaller of an amount of available time implied by the estimate and a maximum time for the interval.

5. The method of claim 1 wherein generating the recommendation includes:
determining whether any of the identified intervals is a first interval associated with a beginning of the future block; and
when any of the identified intervals is the first interval, determining an amount of time available during the first interval, wherein:
when the amount of available time is above a predetermined threshold, the recommendation includes releasing the first interval for booking, or
when the amount of available time is below the predetermined threshold, the recommendation includes closing the operating room during the first interval.

6. The method of claim 1 wherein generating the recommendation includes:
determining whether any of the identified intervals is a last interval associated with an end of the future block,
when any of the identified intervals is the last interval, determining an amount of time available during the last interval, wherein:
when the amount of available time is above a predetermined threshold, the recommendation includes releasing the last interval for booking, or
when the amount of available time is below the predetermined threshold, the recommendation includes closing the operating room during the last interval.

7. The method of claim 6 wherein determining the amount of time available during the last interval includes:
identifying an ending time for a last used interval in the future block, wherein the last used interval is associated with a final scheduled operation in the operating room;
identifying a time difference between the ending time for the last used interval and the last interval; and
adding at least a portion of the time difference to the last interval to increase the amount of available time.

8. The method of claim 1 wherein the one or more models include at least one of a Poisson model and a Negative Binomial model.

9. A computing system for improving operating room utilization, the computing system comprising:
at least one processor; and
at least one memory storing computer-executable instructions that, when executed by the at least one processor, control the computing system to:
retrieve statistics indicative of usage rates for an operating room, wherein the statistics include, for each individual previous time block in a plurality of previous time blocks:
a planned usage rate of the operating room for the individual previous time block;
an actual usage rate of the operating room for the individual previous time block; and
metadata indicate a reason for any difference between the planned usage rate and the actual usage rate;
predict an estimate of unused time for a future time block, wherein predicting the estimate includes:
constructing one or more regression models based on the statistics of usage rates for the operating room, wherein each of the one or more regression models identifies a relationship between the planned usage rate and the actual usage rate;
identifying, from the one or more regression models, a best fit model for the statistics of usage rates for the operating room; and
applying the best fit model to planned future usage rates for the operating room during the future time block; and
generate a recommendation for the unused time in the estimate, wherein the recommendation is based at least partially on an identification of one or more intervals in the future time block associated with the operating room being open in the estimate.

10. The computing system of claim 9 wherein the recommendation includes one or more of:
releasing one or more of the identified intervals for additional procedures in the operation room,
rescheduling one or more procedures during the future block into one or more of the identified intervals to increase an amount of continuously available time during the future block,
rescheduling one or more procedures during the future block into one or more of the identified intervals to reduce down time for operating room personnel during the future block, or
closing the operating room during one or more of the identified intervals.

11. The computing system of claim 9 wherein the computer-executable instructions further control the computing system to send the recommendation to at least of a surgeon, a scheduling manager, an administrator associated with the operating room, a block owner, and a patient.

12. The computing system of claim 9 wherein the computer-executable instructions further control the computing system to:
display the recommendation to a user;
receive an indication, from the user, of an approval or a denial of the recommendation, wherein:
when the indication is the approval, the computer-executable instructions further control the computing system to update the planned usage rates during the future block based on the recommendation; and
when the indication is the denial, the computer-executable instructions further control the computing system to generate a new recommendation for the unused time in the estimate.

13. The computing system of claim 12 wherein the denial includes an indication of one or more reasons for denying the recommendation, and wherein the new recommendation is based at least partially on the one or more reasons for denying the recommendation.

14. The computing system of claim 12 wherein the user is a second user, and wherein the computer-executable instructions further control the computing system to:
receive, from a first user, at least one request for an operating room procedure during the future block, wherein the recommendation is further based at least partially on the request; and
communicate the indication to the first user.

15. The computing system of claim 9 wherein the computer-executable instructions further control the computing system to receive at least one request for an operating room procedure during the future block, wherein the recommendation includes an indication of which of the one or more identified intervals are available to satisfy the at least one request.

16. The computing system of claim 9 wherein:
the operating room is a first operating room, the estimate is a first estimate, and the identified intervals are first identified intervals;
the computer-executable instructions further control the computing system to predict a second estimate the unused time for the future time block in a second operating room; and
the recommendation is further based at least partially on an identification of one or more second intervals in the future time block.

17. A non-transitory computer-readable storage medium storing instructions that, when executed by a computing system, cause the computing system to perform operations for improving operating room utilization, the operations comprising:
retrieving statistics indicative of usage rates for the operating room, wherein the statistics include, for each individual previous time block in a plurality of previous time blocks:
a planned usage rate of the operating room for the individual previous time block;
an actual usage rate of the operating room for the individual previous time block; and
predicting an estimate of unused time for a future time block, wherein predicting the estimate includes:
constructing one or more regression models based on the statistics of usage rates for the operating room, wherein each of the one or more regression models identifies a relationship between the planned usage rate and the actual usage rate; and
applying the one or more regression models to planned future usage rates for the operating room during the future time block; and
generating a recommendation for the unused time in the estimate, wherein the recommendation is based at least partially on an identification of one or more intervals in the future time block associated with the operating room being available in the estimate.

18. The non-transitory computer-readable storage medium of claim 17 wherein the planned usage rate and the actual usage rate each include data at a surgeon level, a surgical service level for a surgical group associated with the operating room, and an entire population level for all staff associated with the operating room.

19. The non-transitory computer-readable storage medium of claim 17 wherein the estimate of unused time for the future time block includes a plurality of sub-estimates each associated with a different level of abstraction for usage of the operating room.

20. The non-transitory computer-readable storage medium of claim 17 wherein the recommendation is further based at least partially on a predetermined required amount of unused time for the operating room.
